(19) United States
(12) Patent Application Publication
(10) Pub. No.: US 2010/0179522 A1
(43) Pub. Date: Jul. 15, 2010

(54) System for endosurgical removal of tumors by laser ablation with treatment verification - particularly tumors of the prostate

(75) Inventors: John Alexander Companion, Newport News, VA (US); Bobby Gale Batten, Williamsburg, VA (US)

Correspondence Address: Bobby G. Batten, 112 Samuel Sharpe, Williamsburg, VA, 23185, US

(73) Assignee: MedSci Technologies, Inc.

(21) Appl. No.: 12/655,623

(22) Filed: Jan. 4, 2010

Related U.S. Application Data
(60) Provisional application No. 61/204,983, filed on Jan. 14, 2009.

(51) Int. Cl. A61B 18/20

(52) U.S. Cl. 606/10

(57) ABSTRACT

The disclosed invention is a unique, patient-friendly, laser-based tumor ablation system for the removal of malignant tumors of the prostate and, with modified delivery systems, may have application for other areas of the human body.

The disclosed invention is an integrated, robotic treatment subsystem that takes advantage of the capabilities of the previously disclosed MedSci Detection, Mapping and Confirmation System, for the purpose of providing a patient friendly system and method for removing tumors detected by said diagnostic system. The invention is a laser-based endosurgical thermal treatment system that utilizes historical cancer mapping data together with real-time ultrasonic and other data to reliably target and control the eradication of cancer conditions. The system contains computer aided robotic control such that control of the boundary, size, position and orientation of the ablated volume of tissue has a tolerance of less than a millimeter. The disclosed system provides multimodal scanning methods for improved identification and localization of detected tumors, including multi-focal tumors. The disclosed system also provides multiple methods for monitoring the successful progress and conclusion of the treatment. The disclosed system provides the capability of closing the created cavity. The disclosed system resides in a subsystem module and when treatment is to be conducted, the treatment module is substituted in place of the previously disclosed ultrasonic diagnostic module of the MedSci system. The subject thermal treatment system meets the challenges confronting the advancement of thermal treatment systems in the search for a highly effective and patient-friendly cancer treatment.

### CROSS-REFERENCE TO PRIOR PATENT AND RELATED APPLICATION

This application claims the benefit of prior U.S. Pat. No. 6,824,516 and the U.S. Provisional Application No. 61/204,983 filed Jan. 9, 2009 for “Endoscopic Laser-Based Tumor Ablation System.”

### INVENTORS

### John Alexander Companion, 344 Walt Whitman Ave., Newport News, Va.

### Bobby Gale Batten, 112 Samuel Sharpe, Williamsburg, Va.

### BACKGROUND OF INVENTION

1. Field of the Invention

This invention relates generally to medical devices.

It relates particularly to the treatment of malignant tumors.

This embodiment relates specifically to the treatment of cancer of the prostate.

This invention lies within the class of medical devices identified as endoscopic surgical systems.

This invention is an endosurgical, Holmium laser based, robotic system that can totally erode and remove tissue volumes containing tumors with process tracking, treatment confirmation and closure, and is particularly applicable to tumors of the prostate.

This invention discloses an integrated, high quality, patient-friendly treatment system that couples with the Diagnostic, Mapping and Confirmation system disclosed in U.S. Pat. No. 6,824,516.

2. Description of Related Art

Prostate cancer is a frequent diagnosis in older males and a plethora of techniques, devices and systems have been developed to address the treatment of such tumors where it is deemed medically desirable to attempt removal or treatment of singular or multi-focal tumors. Many of the existing techniques for treating prostate cancer are intended to treat the entire prostate or significant portions thereof. Such techniques include Brachytherapy, Cryogenic, RF, Magnetostricitve and Ultrasonic, all of which use thermal effects to cause cellular necrosis. The problems with these types of procedures are three-fold: (1) there is no effective way to precisely control the boundary of the treated volume, frequently resulting in incontinence and infertility because the functional structures of the prostate are destroyed or damaged, (2) there is no effective way to know that all of the cancer, or cancers in the case of multi-focal tumors, have in fact been uniformly treated, and (3) therefore, there is no effective way to know if the treatment has been sufficient to ensure necrosis of the entire tumor.

Systems that can address the tumor more directly offer greater opportunity for minimization of collateral damage to non-involved tissues and structures.

Examples of patents, which disclose such systems and related techniques, are described in U.S. Patents:

| | | |
|---|---|---|
| 7,607,440 | Coste-Maniere, et al. | October 2009 |
| 7,108,688 | Jensen | September 2006 |
| 6,676,669 | Charles, et al. | January 2004 |
| 6,441,577 | Blumenkranz, et al. | August 2002 |
| 5,808,665 | Green | September 1998 |
| 5,597,146 | Putnam | January 1997 |
| 5,445,166 | Taylor | August 1995 |
| 6,151,981 | Costa | November 2000 |
| 5,417,210 | Funda, et al. | May 1995 |
| 5,697,939 | Kubota, et al. | December 1997 |
| 7,447,537 | Funda, et al. | November 2008 |
| 7,211,080 | Treat, et al. | May 2007 |
| 6,638,289 | Johnson, et al. | October 2003 |
| 6,132,448 | Perez, et al. | June 2000 |
| 6,572,632 | Zisterer, et al. | June 2003 |
| 6,494,896 | D'Alessio, et al. | December 2002 |
| 6,004,547 | Rowe, et al. | December 1999 |
| 5,372,585 | Tiefenbrun, et al. | December 1994 |
| 6,312,441 | Deng | November 2001 |
| 5,797,849 | Vesely, et al. | August 1998 |
| 6,246,898 | Vesely, et al. | June 2001 |
| 5,201,731 | Hakky | April 1993 |
| 4,955,882 | Hakky | September 1990 |
| 4,694,828 | Eichenbaum | September 1987 |
| 5,061,266 | Hakky | October 1991 |

Notwithstanding the achievements of the referenced inventions, the fact remains that no technique presently exists which provides total, verifiable, precise control over the size and shape of the volume of tissue to be removed, thus they do not permit the reliable avoidance of non-involved tissue for maximum retained functionality. Nor do any of the referenced inventions address the issue of tumor cells being dislodged during the procedure to potentially cause later metastases. Nor do any of the systems provide an integrated capability for both real-time tracking of the procedure process and inspection of the interior of the created cavity replacing the tumor for the presence of residual malignant tissue. Nor do any of the existing systems provide the capability of addressing multi-focal tumors on an individual or group basis. Nor do any of the existing systems provide the capability of accomplishing these activities via a single, needle-like applicator. Nor do any of the existing systems provide control over the eradication procedure to a precision of less than a millimeter for maximum precision in Physician control over the volume of tissue to be ablated.

Every type of prior art has shortcomings that can result in urinary or sexual dysfunction, destruction of noninvolved tissue, and no endoscopic surgical system addresses the known possibility of procedure dislodged tumor cells escaping treatment to cause metastases or recurrence.

With existing technology, because of a lack of precise control over the size and shape of the volume of tissue that is treated to eliminate the tumor, there is frequent collateral damage to tissue not involved with the tumor, which can adversely affect normal functionality of the prostate.

In procedures, such as ultrasound or radio frequency ablation, there is also the question of coverage; i.e. insuring that all of the volume containing the tumor is uniformly affected by the treatment sufficiently to ensure complete cellular necrosis. Likewise, insuring that non-involved tissue is not affected by the treatment is difficult.

None of the prior art provides for real-time confirmation of treatment effectiveness.

None of the prior art provides the use of near IR optical shadow techniques, which can offer improved identification, and localization of multi-focal tumors.

Far more desirable is a system that can deliver the theoretical maximum in precision of tumor eradication so that, if non-involved, the functional structures of the prostate can be maintained. With the disclosed invention there is no question about the boundary or completeness of removal of exactly the tissue volume and shape from the position specified by the Physician, and offers both immediate verification of removal of malignant tissue and mechanisms to eliminate the possibility of cancerous cells being dislodged and escaping to potentially cause later recurrence or metastases.

No prior art provides for the use of a highly precise mechanical control system directing a Holmium laser beam from the side of a rotating tip of a needle-like applicator which offers a pathway to the maximum theoretical efficacy in tumor removal and a practical approach to implementation, as will be disclosed in this application.

### SUMMARY OF THE INVENTION

It is accordingly a primary object of the present invention to obviate the disadvantages presented by systems and processes of the Related Art. This object is achieved and attending benefits are acquired, by the provision of an endoscopic laser ablation subsystem, which is coupled with and takes advantage of the diagnostic and mapping systems disclosed in U.S. Pat. No. 6,824,516.

Following a diagnosis of prostate cancer in a given patient by the MedSci Diagnostic and Mapping System (U.S. Pat. No. 6,824,516), the disclosed invention provides an endoscopic laser ablation system that has the capability of totally removing the detected tumor with precision. The system works with the patented MedSci Transurethral probe, Patient Chair, Electronics and computer systems. The original Transrectal Scanning and Mapping probe subsystem is physically replaced by the complimentary, Transrectal Scanning and Laser Ablation subsystem. The original Mapping subsystem is undocked from the Patient chair and the Transrectal Scanning and Laser Ablation subsystem is docked in its place. The described system houses the same triplex ultrasound scanning system, as did the Diagnostic and Mapping subsystem, as well as the application of Dynamic Elastograpy for enhanced tumor identification and localization.

The purpose of this invention is to provide for reliable, patient-friendly laser ablation of tumors, utilizing the integration of accurate targeting and precise guidance technologies supported by the MedSci Diagnostic System, as well as, real time verification of treatment effectiveness.

Mechanical movements within the support base are similar to the systems used to power the Slaved Biopsy System in the Diagnostic and Mapping subsystem. The movements for the Laser Ablation Needle Applicator are larger, to accommodate the increased functionality, but are functionally analogous. Under the Physician's guidance and using both real time scanning and archived data from the patient's original diagnostic procedure, the computer controlled movement robotically advances a laser ablation applicator needle out of the side of the transrectal probe, through the rectal wall and into the prostate capsule at an angle and vector that will place the tip of the applicator probe just short of the mapped tumor and on a path that is preferably tangential to that tumor.

By following a tangential path for the ablation procedure, only the laser beam enters the tumor and there is no possibility of dislodging cells to cause metastases. The disclosed system is designed to create a cavity, which replaces the volume of tissue containing the tumor, along with an additional surrounding volume, which is a Physician specified margin to ensure total removal of the tumor. Using the tangential approach, the created cavity is a generalized wedge shape with the narrow edge lying along the side of the Laser Ablation Needle. The cavity is created in additive slices, each of which increases the longitudinal size of the cavity.

The cavity is created by projecting a Holmium laser beam onto the tumor tissue through a side port in the rotatable tip of the laser ablation needle. The laser beam is rotated in an arc to sweep over the tumor and vaporizes the tumor tissue at the point of impingement. Each sweep of the beam across the tumor tissue vaporizes a thin layer of tissue. The vapors created by that vaporization action are extracted through the hollow needle via a modulatable vacuum system. The vacuum system operates in concert with an inert gas injection system, such that enough gas is injected into the created cavity to replace the extracted vapors and hold the created cavity open.

The geometry of each successive slice is modulated to enclose the shape and size of the corresponding cross section of the tumor with the specified margins at that axial location. The geometric control afforded by the mechanical elements of the disclosed system is such that the cavity size, shape, and orientation are controllable to sub-millimeter precision. Such precision ensures minimum collateral damage to non-tumor involved tissues and structures. This is important in maintaining maximum normal functionality of the prostate.

The functional elements that control the rotation of the laser ablation needle, the routing of the laser beam to the tip, the routing of the inert gas injection, the routing of the vacuum extraction action as well as the other mechanisms required for all other functionalities of the laser ablation needle itself are housed in a control and routing cassette, so that by controlling the azimuthal, angle, and linear movement of the cassette, the directionality and vector movement of the tip of the laser applicator needle can be brought to bear on a detected tumor, regardless of location, with full functionality.

The disclosed system provides real time monitoring of the creation of the cavity on the computer display screen by showing the outline of the detected tumor, the planned cavity with margins and the actual cavity as it is created, all in superimposed fashion. The image of the cavity boundary is accomplished by taking advantage of the fact that high frequency ultrasound does not propagate significantly in a gas. Nothing shows up to ultrasound as clearly as the boundary of a cavity. The triplex ultrasound scanners can thus see each increment of the creation of the cavity that eradicates the tumor. In this way the Physician observes the planned ablation as it replaces the tumor volume. The designated volume is ablated in axial layers, each being the thickness of the laser beam. The laser ablation needle applicator then advances a step, and the process is repeated until the entire tumor is replaced by a continuous cavity.

Further functionality provided by the disclosed system is the ability to inspect the interior of the created cavity for any residual tumor tissue and re-apply the ablation laser beam to any such tissue that might be found.

The overall process for monitoring and control of the described thermal treatment operations follows. At the beginning, the first step will be to map again in real time the prostate location and cancer area to be treated in relationship to the location of the treatment subsystem utilizing the transurethral and transrectal ultrasonic imaging systems. To expedite this step the system will use the historical detection and mapping data (previously acquired by the MedSci diagnostic system) together with the historical magnetic positioning data and current magnetic positioning input. Having acquired new real-time imaging and compared the screen display of the historical and current images of the cancer, a computer-generated 3-D treatment grid is produced of the tissue volume containing the tumor and the planned treatment safety margins. This will facilitate control of the treatment process.

The time for completion of each eradicating sweep is a function of the selected constant speed rate and the angular distance between the Laser Applicator Needle and the wall of the cavity to be created. Also, the depth of the Holmium laser penetration has been premeasured for various rotational speeds for the needle applicator (i.e. time on target for the laser) thus the computer software can keep track of the tissue volume eradicated vs. planned volume by counting sweeps. Such information, in conjunction with the known spacing of the computer-generated mapping grid, can be utilized by the software to provide guidance for when and how often to apply verification of treatment status with the laser fluorescence capability. These integrated modalities, together with the real-time ultrasonic imaging of the cavity creation, function to provide precise control over the size, shape and orientation of the tumor eradication process with effectiveness verification.

When confronted by a large tumor, the physician can elect to use a Centroid approach for the Laser Ablation needle to pass through the body of the tumor, which allows the ablated cross-section to be a full 360 degrees, or to ablate the tumor sectionally by withdrawing and reinserting the laser ablation needle applicator along a different vector to bring the ablation action to bear on a different area. This can be repeated as needed.

Because using the Centroid approach causes the Laser Ablation Needle to penetrate the body of the tumor there is the possibility of dislodging tumor cells, which could be pushed out of the tumor site and cause metastasis. To eliminate this possibility, the Laser Ablation Needle has the ability to heat the tissue around the penetrating tip of the needle to a temperature that will necrotize any tumor cells, which might have been dislodged and are being pushed by the needle when it penetrates the far side of the tumor. This functionality is preferentially provided by a heating element within the rotating tip to elevate the temperature of the tip causing necrosis of adjacent cells, which would include any malignant cells being pushed out of the tumor by the movement of the needle applicator tip. In general the tangential approach is preferred, as it eliminates this issue.

An alternative embodiment uses the laser ablation needle rotating tip to create frictional heating. The tip of the Laser Ablation Needle is normally rotated to sweep the laser beam over the tissue to be vaporized. The rotational mechanism if speeded up, will cause frictional heating in the tissue adjacent to the rotating tip sufficient to necrotize the tissue. This would be done twice; once as the tip of the needle approaches the far side of the tumor that it is penetrating and again, after the needle has penetrated the far wall to ensure that no tumor cells escape during treatment. This action is under the control of the Physician, and prior to initiating this action the ablating laser is turned off.

In cases where multi-focal tumors are suspected (historical data shows that 25% of prostate cancer patients have more than 2 cancer locations) the described system can apply an augmentation-imaging package to permit the physician to further evaluate the prostate conditions and decide on the best procedure for eliminating the detected tumors. This procedure involves replacement of the transurethral ultrasound probe with a light pipe of similar form and size. A set of spectrally selected LEDs pumps said light pipe with light comprised of selected wavelengths, which are differentially absorbed by the more dense tissue of malignant tumors. Tumors present in the path of said light are therefore backlit relative to a double row of photo detectors in the Transrectal probe, appearing as shadows to the imaging system. By having the light emitted through a narrow window at the moveable tip of the light pipe, that light source can be stepped along the length of the prostatic urethra. The light frequencies used lie in the near infrared region, which are known to penetrate tissues to depths of up to 10 cm. A double row of photo-detectors are mounted in the transrectal probe. They are in fixed position and extend the full length of the transrectal probe. As the light source in the urethra is stepped, the changing geometry will cause the shadow vectors of each tumor to change in a specific pattern. That shadow pattern, arriving at the photo-detectors can be used to calculate the number and locations of multifocal tumors down to a small size. Additionally, that differential absorption by the tumor is known to cause a pressure pulse to be emitted by the tumor, which can be detected by the ultrasound scanners in the transrectal probe and serve as corroboration of malignancy.

Control over the directionality and depth of penetration of the laser ablation beam being projected from the applicator needle in conjunction with the precise positioning and vector movement enables the system to address detected multi-focal tumors individually, as groups, or by ablating whatever volume is necessary up to a full prostatectomy.

Control over the boundary of the ablated area is such to permit the preservation of non-involved areas, which could include the prostatic capsule boundary, the urethra and the upper and lower sphincters.

Further, as described in prior U.S. Pat. No. 6,824,516, the system provides the option of filling the created cavity with an inert gel material (which may be loaded with appropriate drugs). Alternatively, the cavity can be collapsed by using the vacuum system. Tissue adhesive can then be used to seal it closed.

By virtue of the use of mechanical positioning and drive systems that are based on well developed technology in the machine tool industry, accuracy of control of the laser beam movement is in the sub-millimeter range at all times. Thus providing a high safety factor in the system control capability. The described system can remove a specific volume of material, of a specific shape, from a specific location. In this case the material is tumor tissue, but it is essentially a machining operation, capable of high precision.

### BRIEF DESCRIPTION OF THE DRAWINGS

The illustrations consist of drawings pertaining to both the disclosed treatment invention and a previously patented MedSci System for Diagnosis, which provides support for said treatment system. The first 12 drawings provide the background necessary to understand discussion of the details of the invention depicted in the drawings and its integration with the referenced diagnostic system. Integration of the herein-disclosed treatment system with the previously patented diagnostic system supports the functionality and capabilities described for the treatment system. For example, the disclosed treatment system utilizes identical ultrasonic imaging configurations to those disclosed in MedSci U.S. Pat. No. 6,824,516 for targeting and tracking of the treatment process.

FIG. 1A/B/C Patient Chair—A sectional schematic showing 3 views of the Patient Chair as described in previous MedSci U.S. Pat. No. 6,824,516. For use in the current disclosure, there are no differences. All functionality and features of the previous disclosure accrue to the current application.

FIG. 2 Overall supporting system elements, physical relationship—A sectional schematic view detailing the relationship of the herein disclosed Laser Ablation System module as it is coupled to the Patient Chair and the Electronics Tower as described in previous MedSci U.S. Pat. No. 6,824,516. All functionality and features of the previous disclosure accrue to the current application.

FIG. 3 Interchangeability of the Laser Ablation System module—A schematic view of the interchangeability of the Laser Ablation System module as disclosed herein, with the Mapping and Diagnostic module as disclosed in MedSci U.S. Pat. No. 6,824,516. For clarity only the outlines of the various elements are shown, depicting the geometric relationships of the functional assembly of the three elements to accomplish the disclosed functionality. Those three elements are: (1) the patient chair, (2) The electronics tower, and (3) laser ablation system module.

FIG. 4 Laser Ablation System module showing rocking capability—A sectional, schematic of the functional assembly of the present disclosure, showing the rocking capability of the Laser Ablation System module relative to the Patient Chair for the purpose of properly aligning the movement path of the Transrectal Probe into the patient's rectum as recorded during the previous diagnostic procedure.

FIG. 5 Transrectal probe optical and pressure sensors—A sectional, schematic view of the mounting of the Transrectal probe on the Laser Ablation System module, showing the location of the Upper and Lower video cameras which are used to facilitate the Physician's control over the placement of the Transrectal Probe into the patient rectum by showing him/her real time views of the patient and the Transrectal Probe from above and below the probe. The illustration also shows the Probe tip camera, which both enhances the views shown the Physician during entry movement and also permits the examination of the interior of the rectum after placement. In addition this illustration shows pressure sensors located on the upper and lower surfaces of the Transrectal Probe tip, the outputs of which are displayed for the Physician so that by controlling the movement angle to balance the pressures, the anal entry can be as centered as possible for patient comfort.

FIG. 6 Screen displays for data from the cameras and pressure sensors of the Transrectal Probe, as well as historic and current vertical angle displays—A sectional schematic view of the screen display produced by the instrumentation described in FIG. 5. Said displays are in the same relationship as the actual sensors for more intuitive assessment. The individual display sizes and styles can be rearranged as the Physician finds most useful.

FIG. 7. Transrectal probe at the proper angle for entering anus—A sectional, schematic, anatomical view showing the Transrectal probe angled properly for entry and with the Tip pressure sensors engaging the walls of the anus at the start of the insertion process.

FIG. 8 Transrectal probe in place within rectum for prostate imaging procedure and showing optical view of upper rectum/lower colon. —A sectional, schematic, anatomical view, showing the Transrectal probe in place within the rectum. The upper portion of the rectum is shown being illuminated and inspected by the Probe Tip camera.

FIG. 9 Transrectal probe in place within rectum showing water injection to supply an ultrasound medium. —A sectional, schematic, anatomical view showing the patient rectum being flooded by water, injected from a port on the lower, proximal end of the Transrectal probe. This action is as described in previous U.S. Pat. No. 6,824,516.

FIG. 10 Major ultrasound scanning elements of the Transrectal and Transurethral probes in preferred procedure start position. —A sectional, schematic, anatomical view showing the major ultrasound scanning elements of the Transrectal and Transurethral probes, as they will be at the initiation point of the ablation planning scan procedure. This portion of the procedure does not differ from the previous U.S. Pat. No. 6,824,516 and serves to confirm positioning of the detected tumor relative to the probes. All functionality and features of the previous disclosure accrue to the current application.

FIG. 11 Layout of the dual ultrasound scanners of the transrectal probe—A sectional, schematic view of the dual ultrasound scanners of the transrectal probe as they pass on either side of the Laser Ablation Needle Applicator upper pivot within the Transrectal probe. The physical arrangement does not differ from the previous Patent description of the same subsystem. All functionality and features of the previous disclosure accrue to the current application.

FIG. 12. Overlapping scan fields of the dual Transrectal and Transurethral Ultrasonic scanner subsystems. —A sectional, schematic view of the overlapping scan fields of the Transrectal and Transurethral Ultrasonic scanner subsystems. The physical arrangement does not differ from the previous MedSci U.S. Pat. No. 6,824,516. All functionality and features of the previous disclosure accrue to the current application.

FIG. 13 Major subsystems of the Laser Ablation System module—A schematic view showing the major subsystems of the disclosed Laser Ablation System module in their functional relationship. This includes: all of the movement mechanisms associated with targeting and delivery of the laser ablation treatment, as well as the optical subsystems.

FIG. 14 Mechanical movements of the Laser Ablation System module—A schematic view of the mechanical systems of the Laser Ablation System module, within the enclosure. Shown are: The moveable frame surmounted by the vertical movements (shown in greater detail in FIG. 13) the support structure holding the azimuthal movement at a neutral angle. The vector movement is mounted to the top of the rotary movement. The vector movement holds the linear movement which controls the extension and stepping of the Control and Routing Cassette which provides direct control of the various functions associated with the ablation, confirmation and closure actions of the disclosed invention. (Details of said cassette will be shown in later Figs.) The Control and Routing Cassette is connected to the Laser Ablation Generator and to Fluorescence Confirmation systems via fiber optic cables.

FIG. 15 Laser Ablation Needle Applicator—An overall view of the Laser Ablation Needle Applicator, illustrating all of the movement associated components.

FIG. 16 Rotatable tip of the Laser Ablation Needle Applicator—A sectional, perspective view of the rotatable tip of the Laser Ablation Needle Applicator showing the Ablation laser beam emerging from the distal opening of the central lumen of the drive shaft, being reflected off the 45-degree mirror mounted in the rotatable tip and emerging from the side port of said tip to impinge on the tumor tissue to be ablated.

FIG. 17 Control and Routing Cassette layout—Sectional, schematic views of the details of the Control and Routing Cassette, showing the internal components, passageways and ports that provide the required functionality.

FIG. 18 Control and Routing Cassette configured for Ablation procedure—A sectional, schematic view of the Control and Routing Cassette with the fiber optic connection to the Ablation Laser Generator, the Optical Switch in the ablate position and the pathway of the Ablation Laser beam through the Cassette and being directed at 90 degrees to the axis of the Laser Ablation Needle Applicator.

FIG. 19 Control and Routing Cassette with Vacuum Extraction and Inert Gas Injection Systems highlighted—A sectional, schematic view of the connection and flow routing between the Control and Routing Cassette and both the Vacuum Extraction System and the Inert Gas Injection System.

FIG. 20 Detail of drive shaft showing commutator for electrical power transfer to drive shaft—A sectional, schematic view of the commutator, which supplies electrical power to a heater unit mounted in the Rotating Tip of Laser Ablation Needle Applicator. Power is transferred via redundant pathways on the surface of the Driveshaft from the window area, which permits injected inert gas flow from the Forward Chamber of the Control and Routing Cassette to the distal end of the driveshaft, where it is transferred to the heater.

FIG. 21 Laser Ablation Needle Applicator tip showing heater and electrical power routing from drive shaft—A sectional, schematic of the pickup of the transferred electrical power from the drive shaft and the connections to the heater within the Laser Ablation Needle Applicator tip.

FIG. 22. Control and Routing Cassette configured for Fluorescence Verification of treatment—A sectional, schematic of the Control and Routing Cassette with the fiber optic connection to the Fluorescence Ablation Verification system shown, and with the Optical Switch in the verification position.

FIG. 23A/B Tangential and Circumferential Ablation patterns—A sectional, schematic, anatomical view of the Ablation Laser beam being rotated through an arc (23 a) for a Tangential pattern ablation and the Ablation Laser beam being rotated through a 360 degrees (23 b) for a Centroid ablation pattern.

FIG. 24A/B/C/D Progressive erosion of cross-sectional segment of tumor showing expanding geometry as the ablation zone moves away from the energy source—A sectional, schematic, anatomical view showing the process of swept radial erosion used to form each cross-sectional cavity layer of the creation of a conjoined cavity, which will replace a mapped tumor/margin volume. The erosion is shown at 4 stages of growth.

FIG. 25 Radial Wedge cavity creation from conjoined cross-sectional cavities—A sectional, schematic, anatomical view of the stepwise creation of a generalized web shaped cavity from a Laser Ablation Needle Applicator pathway, which is tangential to a tumor. This causes less distortion of the tumor and permits smaller margins.

FIG. 26A/B Needle distortion of small tumor—A sectional, schematic, anatomical view showing that the penetration of the tip of the Laser Ablation Needle Applicator into a small tumor will cause it to distort, which will throw off the planned ablation pattern.

FIG. 27 Transrectal and Transurethral Ultrasound Scanning systems providing ablation planning—A sectional, schematic, anatomical view showing the Transrectal and Transurethral Ultrasound Scanning systems moving in concert to scan the entire volume containing a detected tumor, to provide a 3-D image for the Physician to use in planning the ablation pattern for eradication of the tumor.

FIG. 28 Laser Ablation Needle Applicator beginning tangential cavity ablation process with ultrasound tracking—A sectional, schematic, anatomical view showing the Laser Ablation Needle Applicator inserted into the Prostate along the planned tangential pathway and creating the second cavity layer of the planned cavity, joined to the already created first layer. This also shows the Transrectal and Transurethral Ultrasound Scanning Systems monitoring the cavity creation.

FIG. 29A/B Fluorescence Verification System showing cancer residue and reapplication of Ablation Laser to that area—A sectional, schematic, anatomical view showing the application of optical energy from the Fluorescence Verification System to the interior of the created cavity (29 a). Unablated residual malignant tissue will produce a characteristic reflection back to the system. 29 b shows the reapplication of the Ablation Laser beam to remove the residue.

FIG. 30A/B/C Using the tip heater to necrotize any possible dislodged tumor cells to prevent secondary metastases—A sectional, schematic, anatomical view showing the application of heat from the internal heater in the Ablation Laser Applicator Needle tip to necrotize any malignant cells that may be dislodged by the Needle Tip when using a Centroid approach to a tumor ablation. FIG. 30 a b c shows the tip advancing through the tumor during the ablation procedure and necessarily penetrating the far wall of the tumor. Heat from the tip is sufficient to necrotize any malignant cells that are being dislodged, carried or pushed by the tip to ensure they are destroyed before they can escape to potentially cause a secondary metastasis.

FIG. 31 Laser Ablation Needle Applicator applying Centroid approach for ablation—A sectional, schematic, anatomical view showing a tumor being ablated using a Centroid approach.

FIG. 32 Skewed ablated cavity creation to match skewed tumor—A sectional, schematic, anatomical view showing that the sequential layers of the created cavity can be skewed to ablate a large oddly shaped tumor using a single insertion of the Laser Ablation Applicator Needle.

FIG. 33. Transurethral Light pipe and optical sensors in Transrectal probe—A sectional, schematic, anatomical view showing light pipe inserted into the Transurethral Catheter and the arrangement of the parallel rows of optical sensors within the Transrectal probe.

FIGS. 34A/B Optical illumination of prostate tissue and shadows cast by tumors onto optical sensors—Sectional, schematic, anatomical views: 34A is a schematic showing a cross section of the prostate with the Transurethral Ultrasound Scanner in place, as it is geometrically related to the Transrectal probe with the contained double row of optical sensors. Small multi-focal tumors may not be prominent in an ultrasound view. 34B shows the Transurethral Ultrasound Scanner replaced by a light pipe. Optical illumination of prostate tissue produces higher contrast and causes small tumors to produce shadows, which makes them more prominent.

FIG. 35 Change of shadow angle resulting from illumination source movement and pickup by multiple optical sensors—Sectional, schematic, anatomical views showing that as the light pipe is advanced through the prostatic urethra, relative to the optical sensors, the shadows cast by the tumors will vary in their impingement points on the optical sensors according to their geometric relationship to the position of the light source and the dual optical sensor rows. Light source position progression is from 35 A through 35 D.

FIG. 36 Controlled directionality of ablation to address multi-focal tumors—A sectional, schematic, anatomical view illustrating controlled directionality of ablation being used to address individual multi-focal tumors.

FIG. 37 Optional use of the Vacuum System to collapse the created cavity after tumor eradication—A sectional, schematic, view of the connections of the Control and Routing Cassette to permit the use of the Vacuum System to collapse a created cavity at the successful conclusion of an ablation procedure. Tissue adhesive is injected to keep the cavity adhered for healing. While this procedure does not differ substantially from the system disclosed in U.S. Pat. No. 6,824,516, routing is modified to utilize the routing and control cassette.

FIG. 38. Optional use of Gel fill of the created cavity after tumor eradication—A sectional, schematic, view of the connections of the Control and Routing Cassette to permit the injection of anti-cancer drug carrying gel to fill the created cavity and promote healing. While this procedure does not differ substantially from the system disclosed in U.S. Pat. No. 6,824,516, routing is modified to utilize the routing and control cassette.

### DETAILED DESCRIPTION OF THE INVENTION

Following is a listing of elements constituting the system of the present invention, along with their corresponding reference numerals, as employed in the accompanying drawings.
1 overall patient chair
2 chair base
3 elastography belt
4 leg rests
5 back rest
6 angle adjustment
7 detecting and mapping subsystem moveable base
8 hip fences with locks
9 transrectal laser ablation subsystem
10 laser ablation subsystem moveable base
11 chair vertical movement
12 joystick movement control for laser ablation subsystem
13 electronics tower
14 touch control screen
15 information display screen
16 transurethral subsystem mechanical movements
17 transurethral subsystem position adjustment mechanism
18 interlocks
19 bellows cover
20 forward vertical jack A/B (pair)
21 aft vertical jack A/B (pair)
22 fluorescence verification system, comprised of fluorescence illuminator 112, return signal splitter 111 and fluorescence detector 113.
23 ablation laser generator
24 A/B transrectal ultrasound scanner drive mechanism
25 A/B transrectal ultrasound scanner drive cable
26 laser movement support bracket
27 azimuthal movement
28 vector movement
29 extensional movement
30 transrectal probe
31 upper camera and support post
32 lower camera
33 upper transrectal probe pressure sensor
34 lower transrectal probe pressure sensor
35 transrectal probe tip camera
35A tip camera illuminator
36 screen display from lower camera
37 screen display showing current vertical angle of transrectal probe
38 screen display showing reference angle from diagnostic procedure
39 screen display from upper camera
40 screen display from lower transrectal probe pressure sensor
41 screen display from upper transrectal probe pressure sensor
42 screen display from transrectal probe tip camera
43 anus
44 prostate
45 rectum
46 urinary bladder
47 lower abdomen
48 water injection port
50 inner support cone
51 transrectal probe backbone
52 A/B transrectal ultrasound scanner elements
53 transurethral ultrasound scanner probe/catheter
54 A/B transrectal ultrasound scanner magnetic markers
55 transurethral ultrasound scanner element
56 transurethral magnetic marker
58 needle pivot
59 laser ablation needle applicator
60 transrectal probe cover
61 A/B transrectal ultrasound scanner movement guides
62 example of scan zone of transurethral ultrasound scanner element
64 A/B example of scan zones of transrectal ultrasound scanner elements a-b
66/67 fiber optic cables
68 rotating laser ablation needle tip
69 needle applicator side port
70 annular injection slot
71 non-rotating needle shell
72 needle support boss
73 aft shoulder of tip drive shaft
74 exposed grooved region of tip drive shaft
75 rotary drive gear
76 base of tip drive shaft
77 forward shoulder of tip drive shaft
78 45-degree mirror in rotating laser ablation needle tip
79 mirror support post
80 central cavity of rotating tip
81 central-axial lumen of tip drive shaft
82 rotating tip drive shaft
83 control and routing cassette
84 drive chamber
85 rotary drive motor/encoder
86 motor drive gear
87 sealed rotary bearings
88 A/B/C/D fiber optic connectors
90 optical switch with mirrors 90A and 90B
91 optical switch chamber
92 optical switch position mechanism
93 A/B rotating tube optical pathways
94 forward port
95 vacuum port
96 inert gas system
97 gas modulating valve
98 vacuum system
99 vacuum modulation valve
101 forward chamber
102 A/B aft pair of commutator brushes
103 A/B forward pair of commutator brushes
104 A/B paired driveshaft conductor segments
105 A/B paired driveshaft conductor segments
106 A/B/C/D axial surface slots on driveshaft
107 A/B paired conductors in rotating tip
108 A/B paired conductors in rotating tip
109 tip heater
111 signal splitter
112 fluorescence illuminator
113 fluorescence detector
121 mapped tumor
122 physician specified margin
125 planned track for ablation
126 planned segmental ablation cavities
127 created cavity segment
128 joined created cavity
129 example of progressive erosion of single segments of a mapped tumor
131 example of residual malignant tissue detected
132 example of needle tip applying heat to surrounding tissue.
133 example of necrotized area after procedure conclusion and needle withdrawal
140 optical emitter moveably placed within transurethral catheter
141 A/B row of optical detectors (paired)
142 example of multi-focal group of tumors
143 tissue adhesive source
144 redirect valve
145 joystick speed control
146 joystick movement increment button
147 example of Holmium laser beam
148 example of overlap of ultrasound scan zones within the prostate
149 example of fluorescence stimulating illumination propagating from side port
150 prostatic urethra
151 example of small, optically dense tumor
152 example of illumination from optical emitter penetrating prostate tissue and impinging on optical detectors
153 example of shadows cast by optically dense tumors on optical detectors.
154 linear movement for transrectal ultrasound scanner elements

Referring now to the drawings; in order to clarify the relationships between the various subsystems of the present invention and how they are used in conjunction with the previous “SYSTEM FOR EXAMINING, MAPPING, DIAGNOSING AND TREATING DISEASES OF THE PROSTATE” (U.S. Pat. No. 6,824,516 assigned to MedSci Inc.), a detailed description is broken down into the following sections:

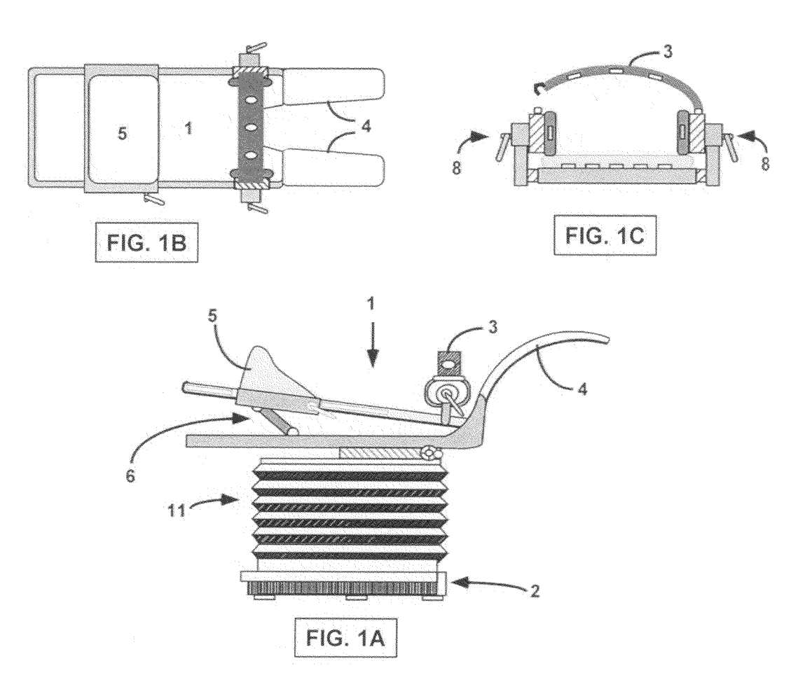

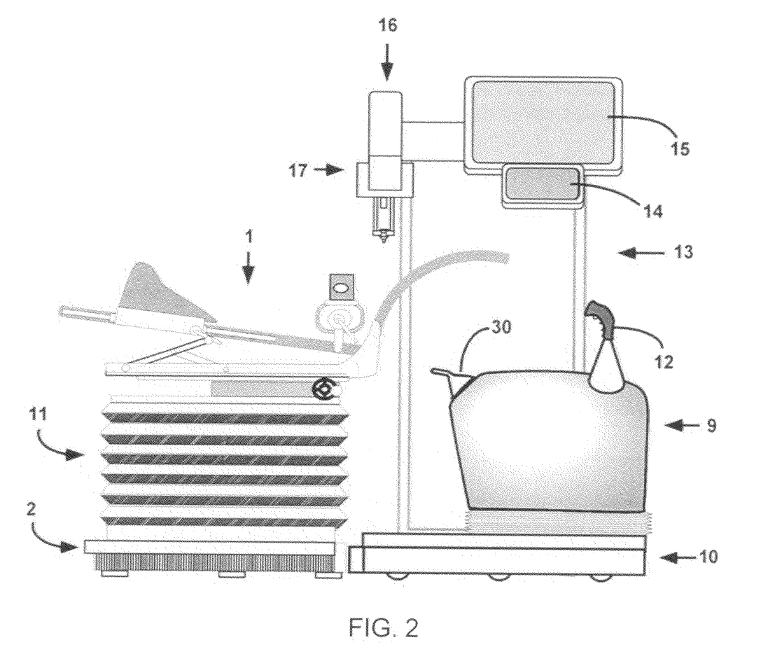

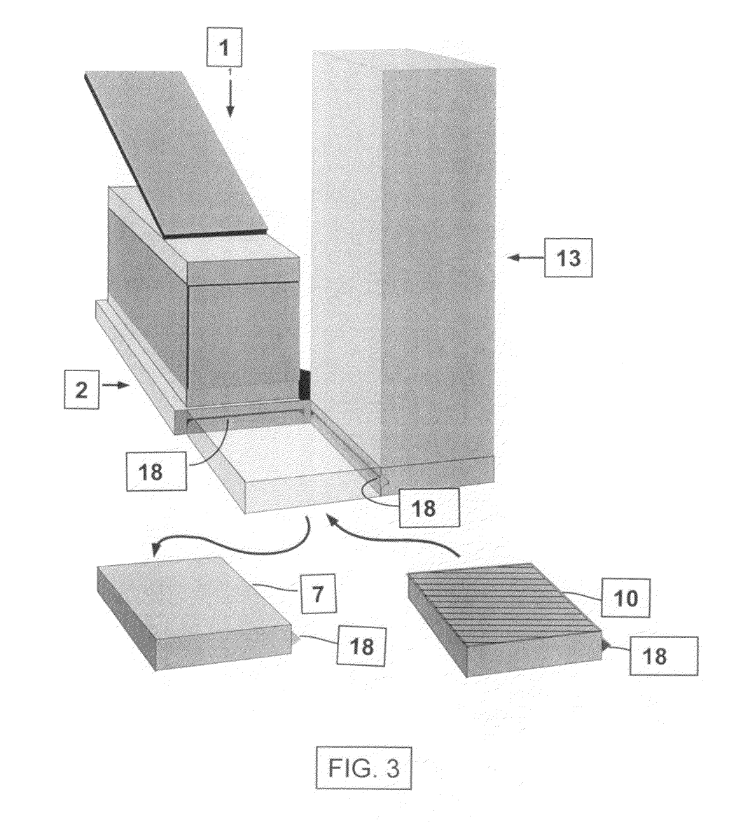

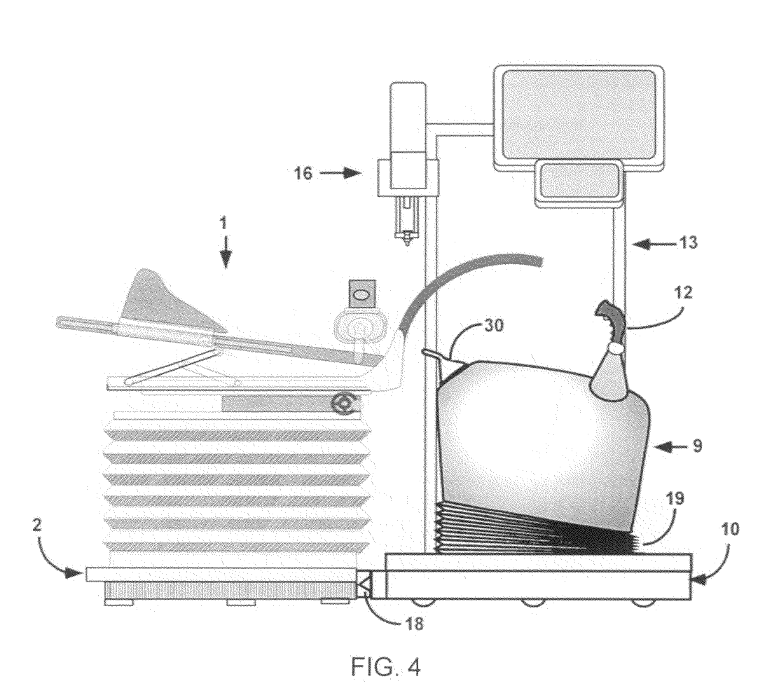

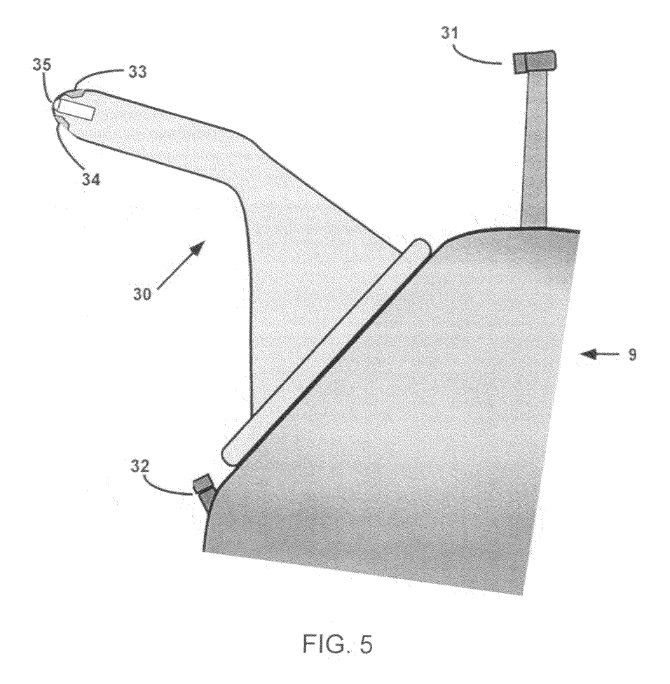

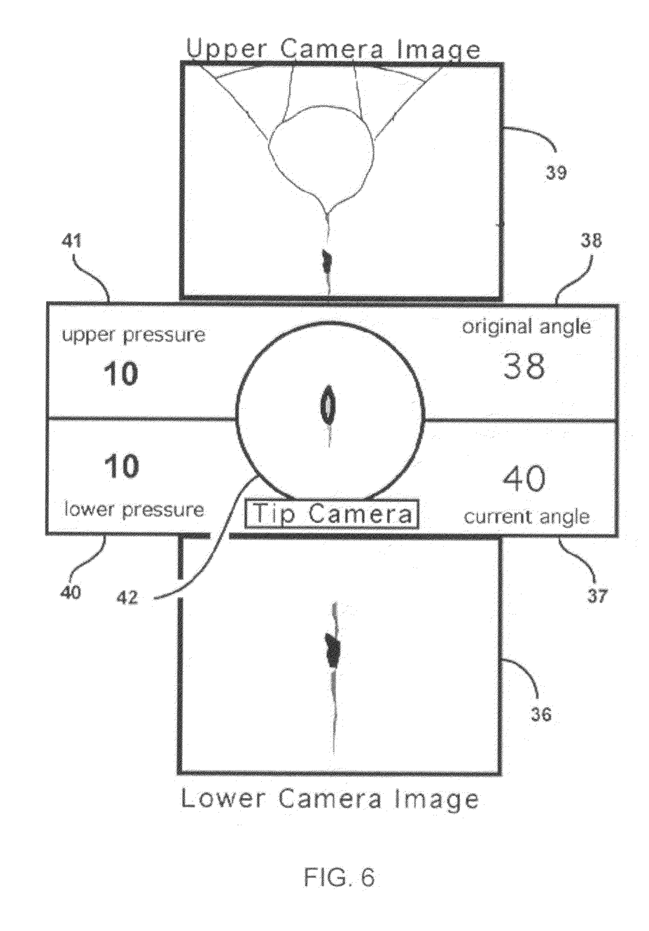

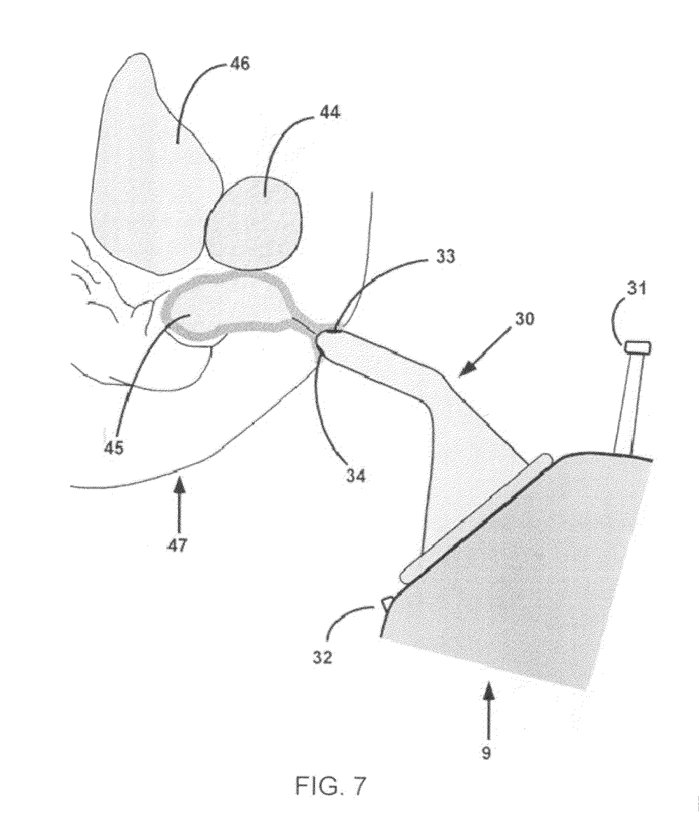

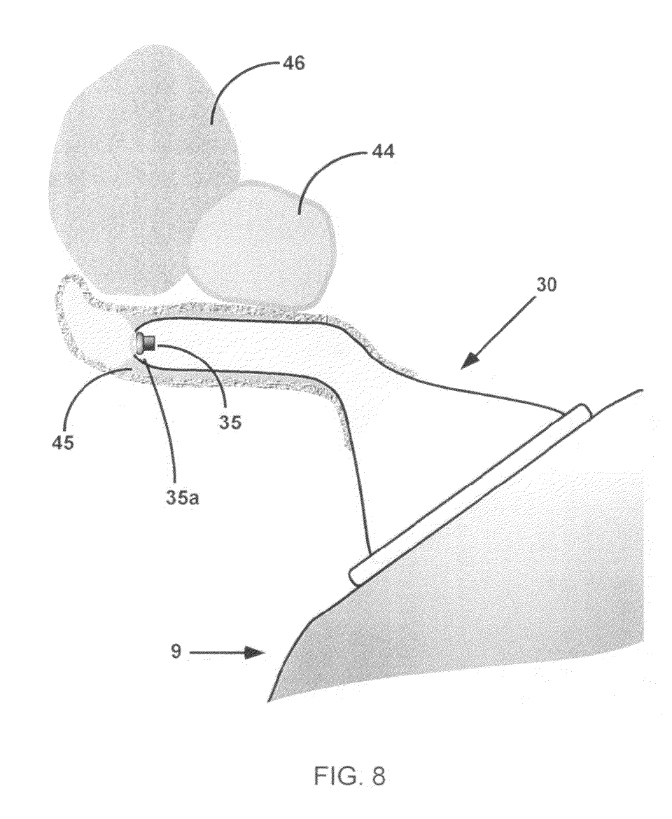

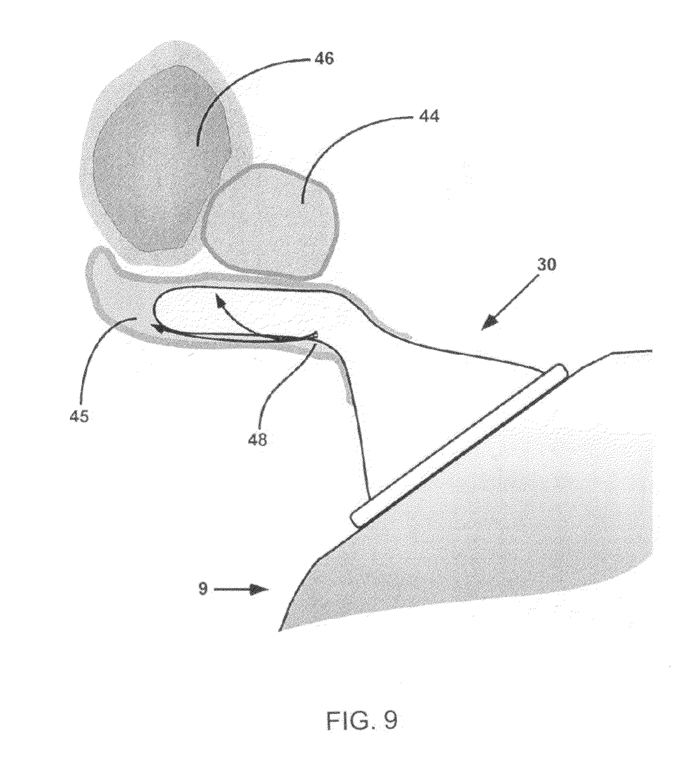

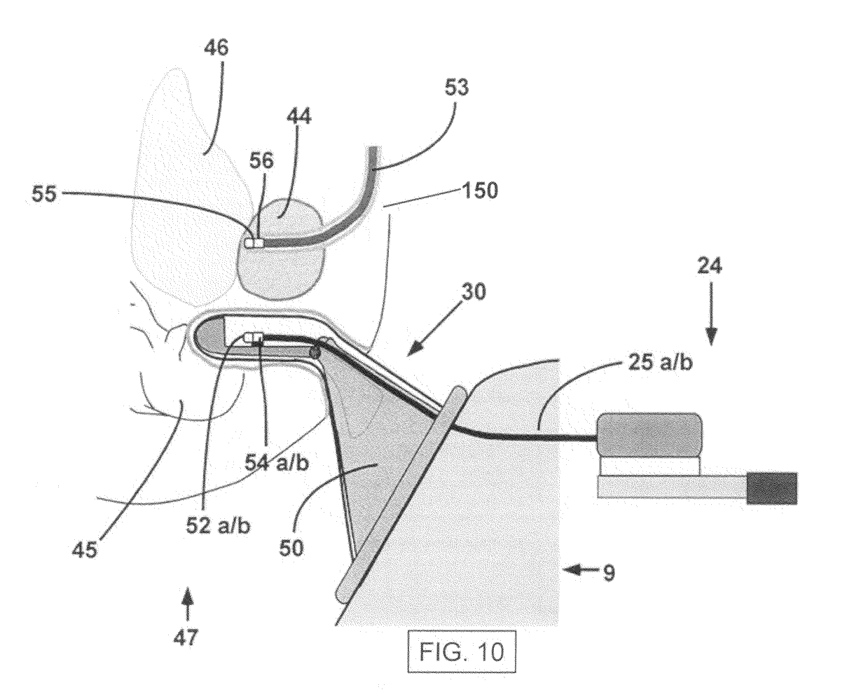

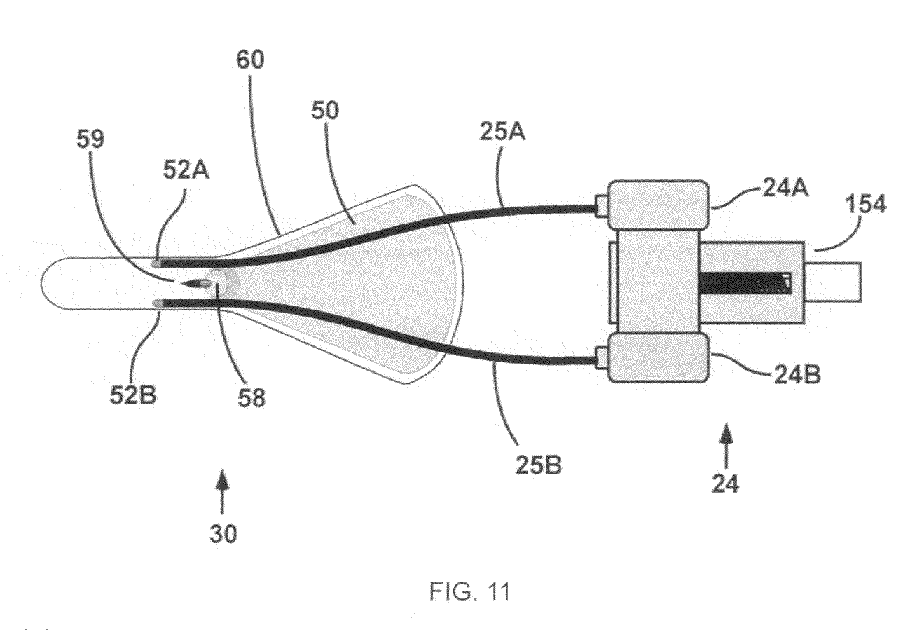

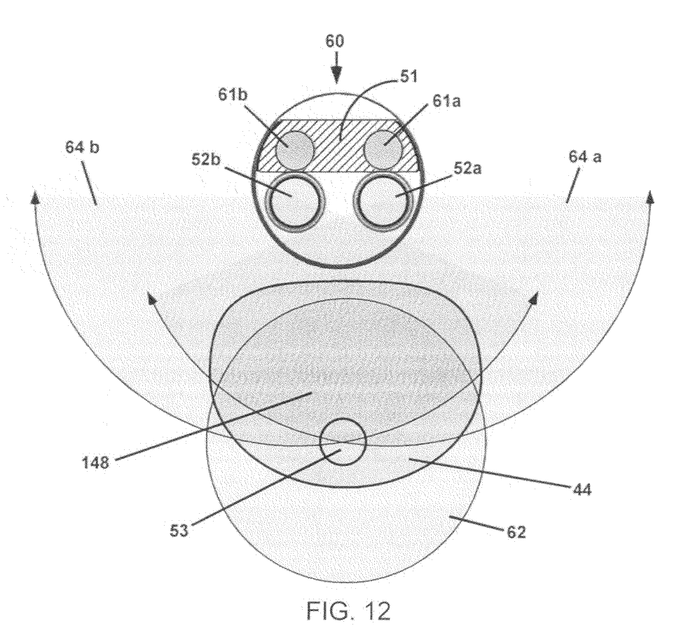

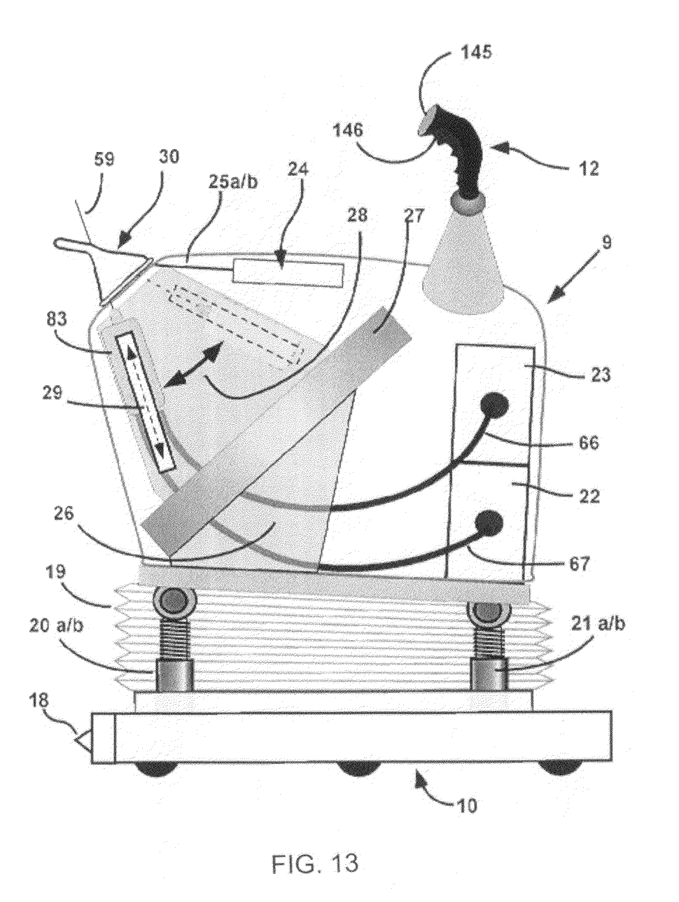

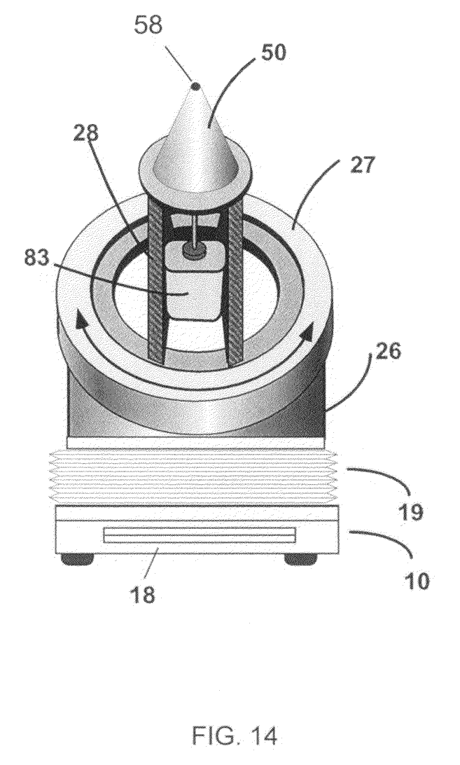

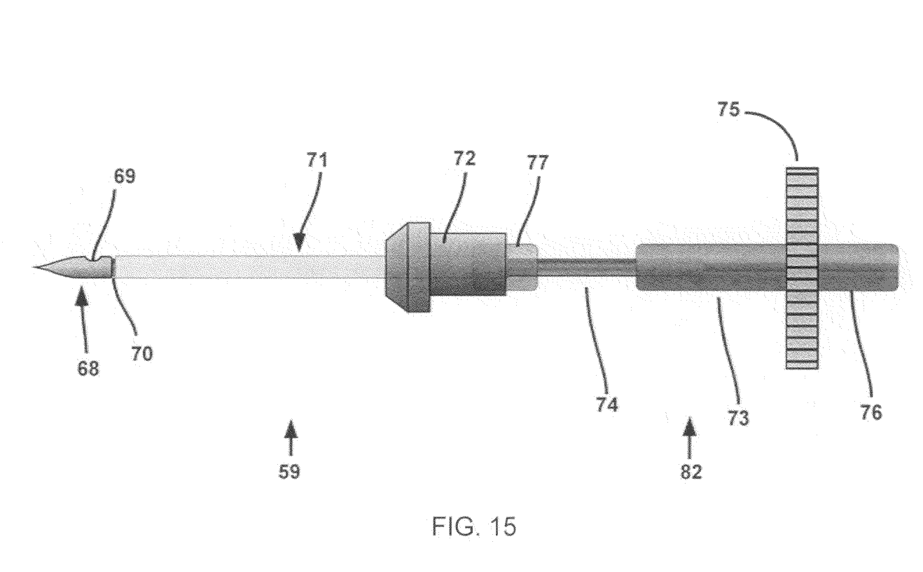

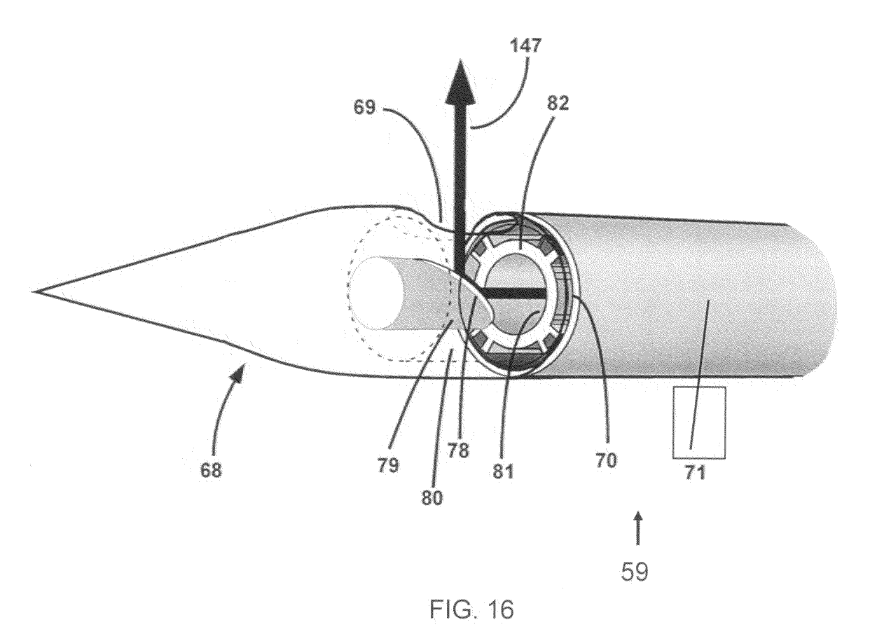

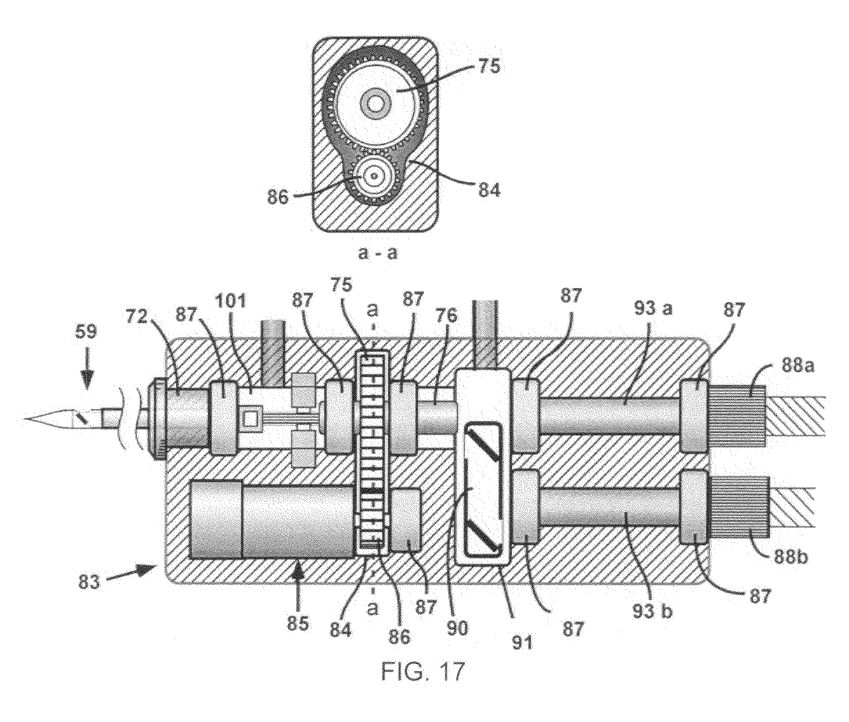

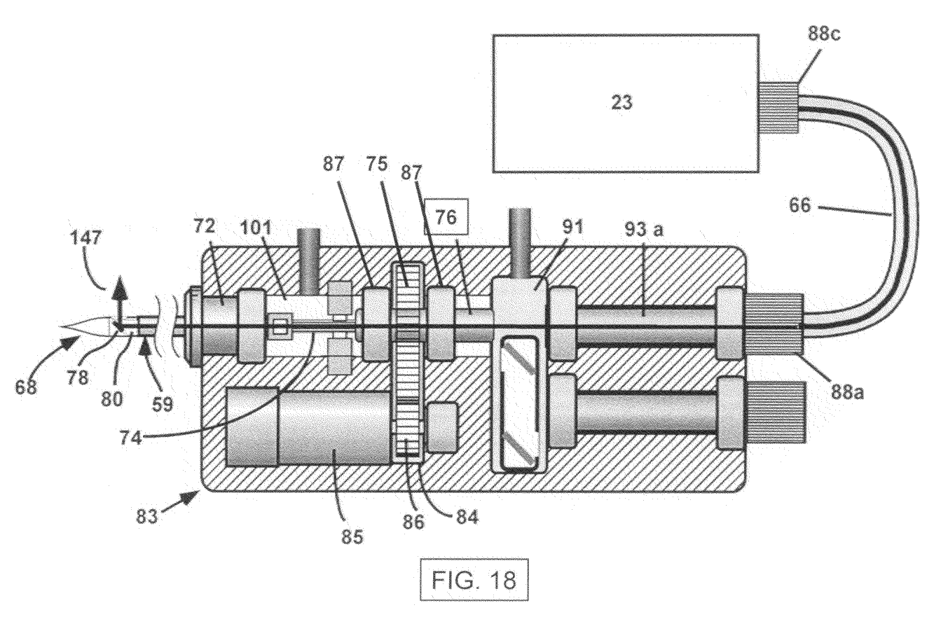

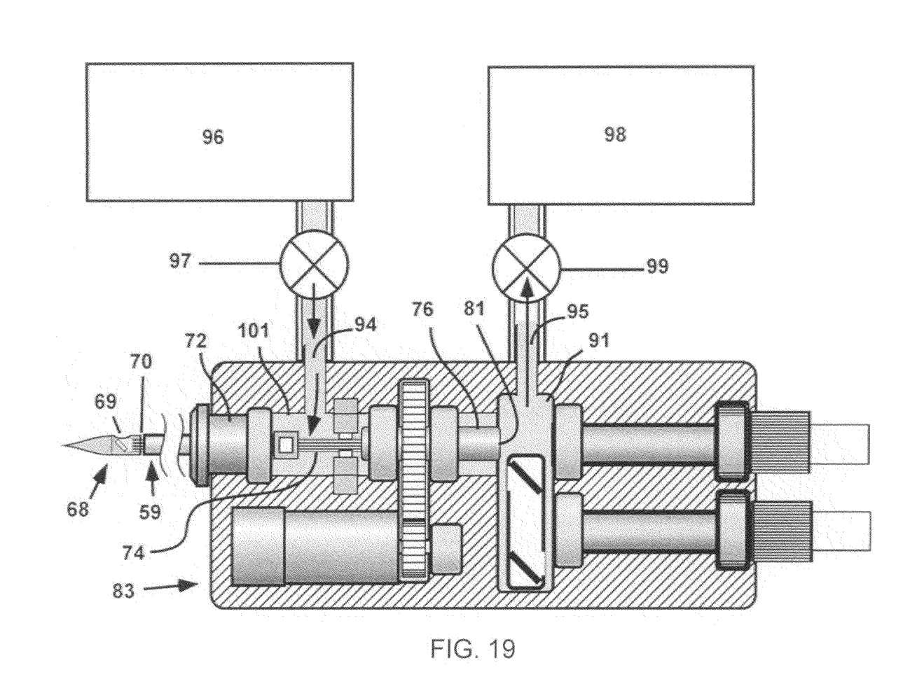

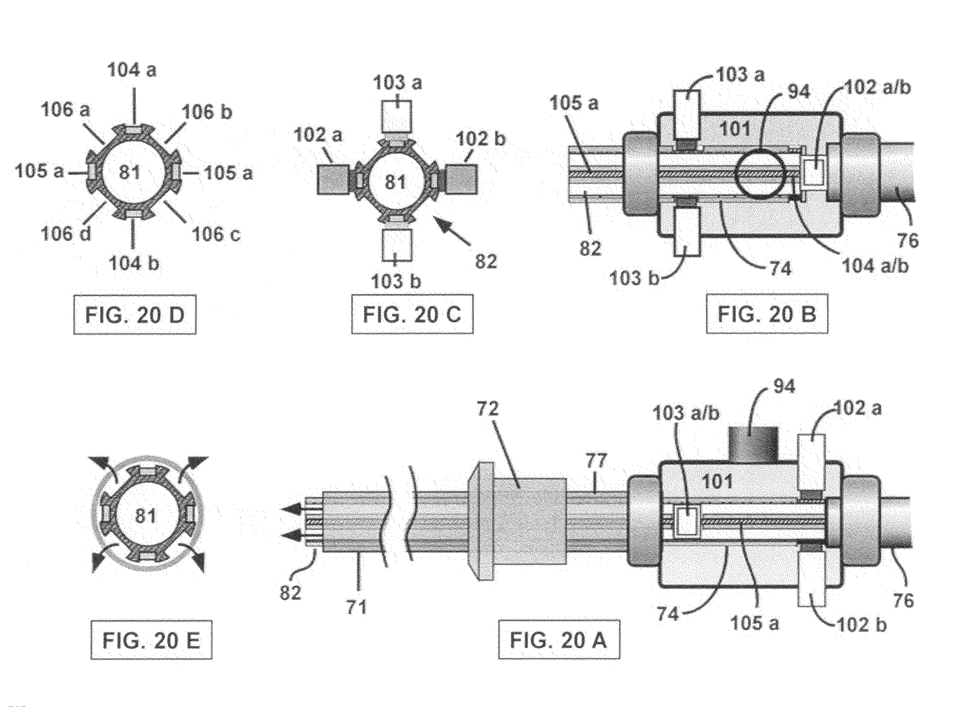

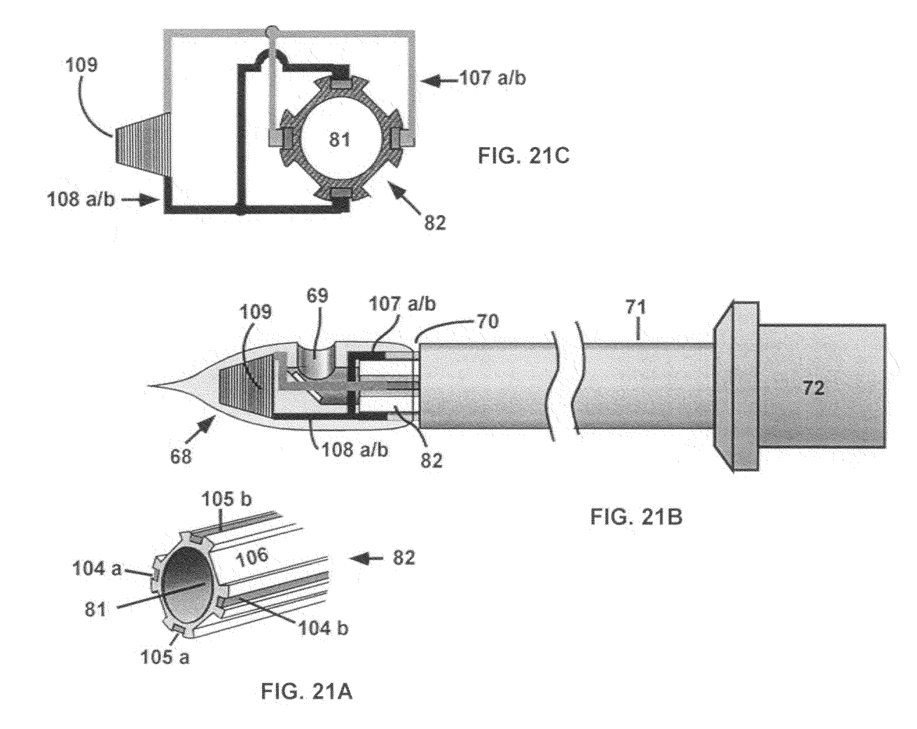

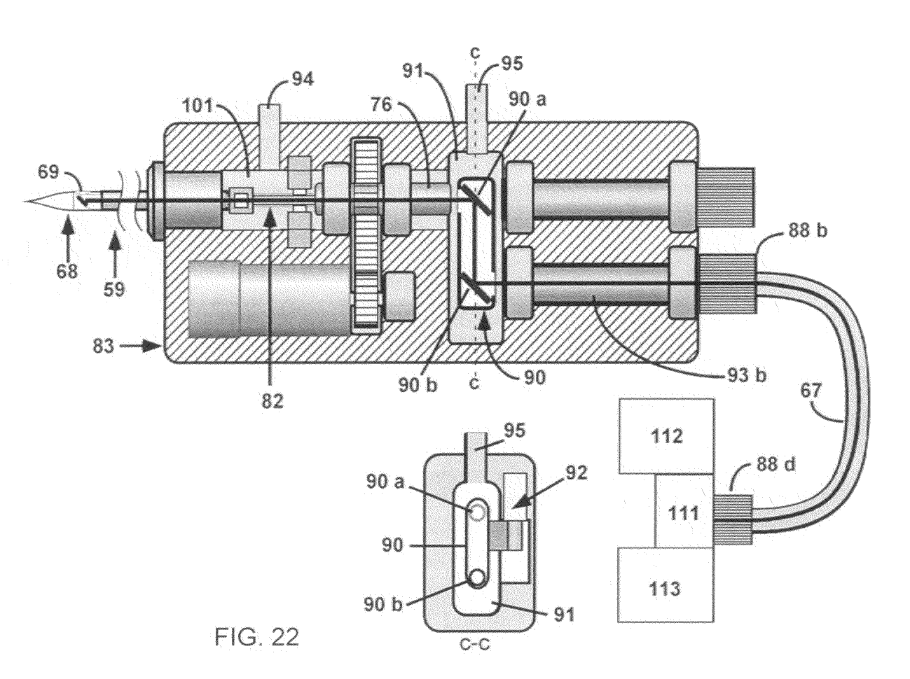

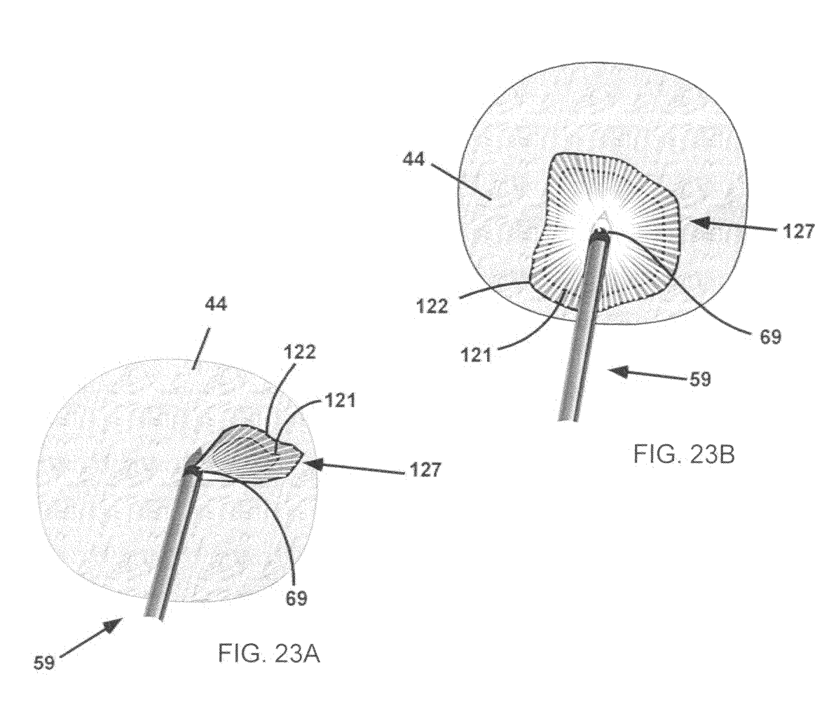

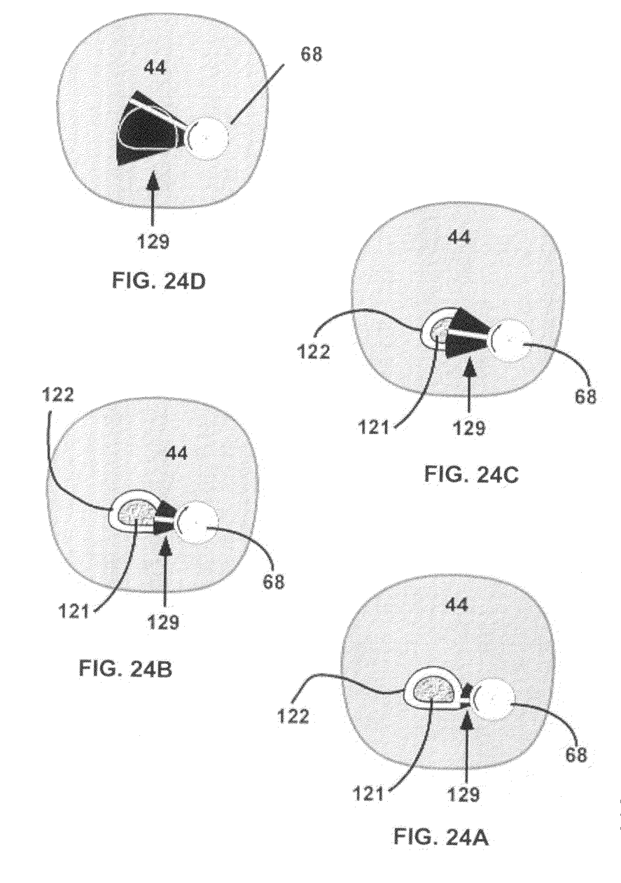

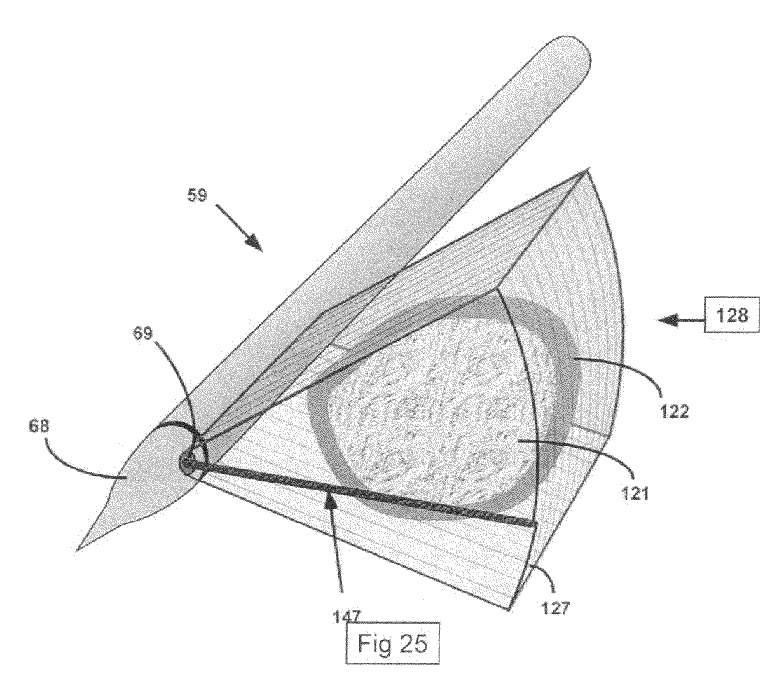

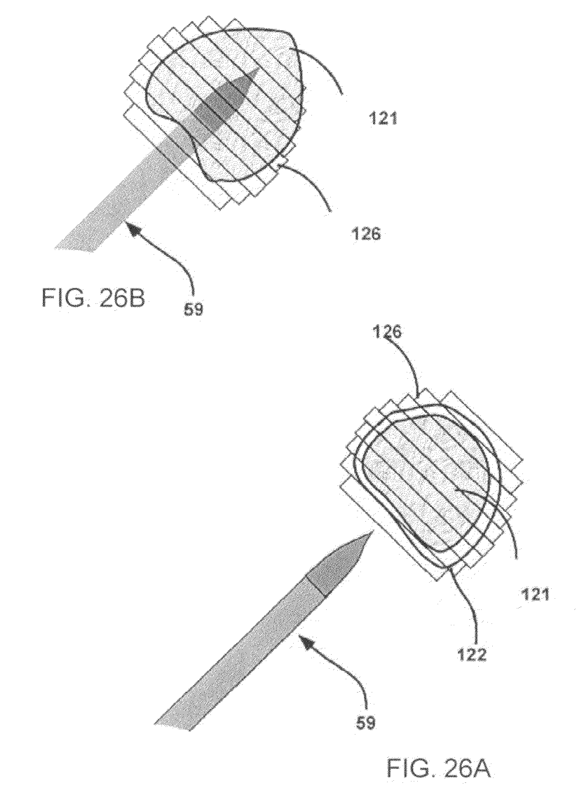

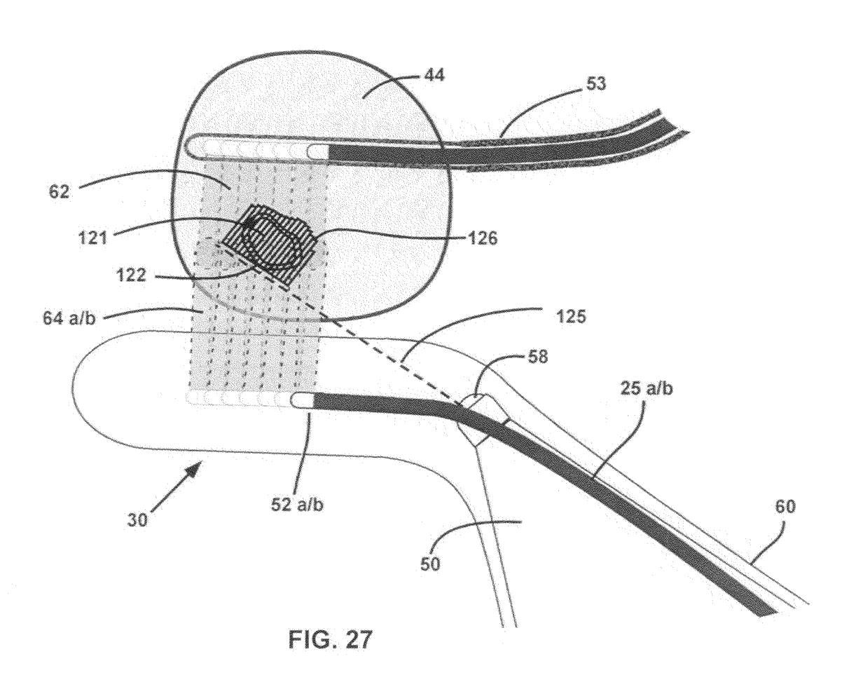

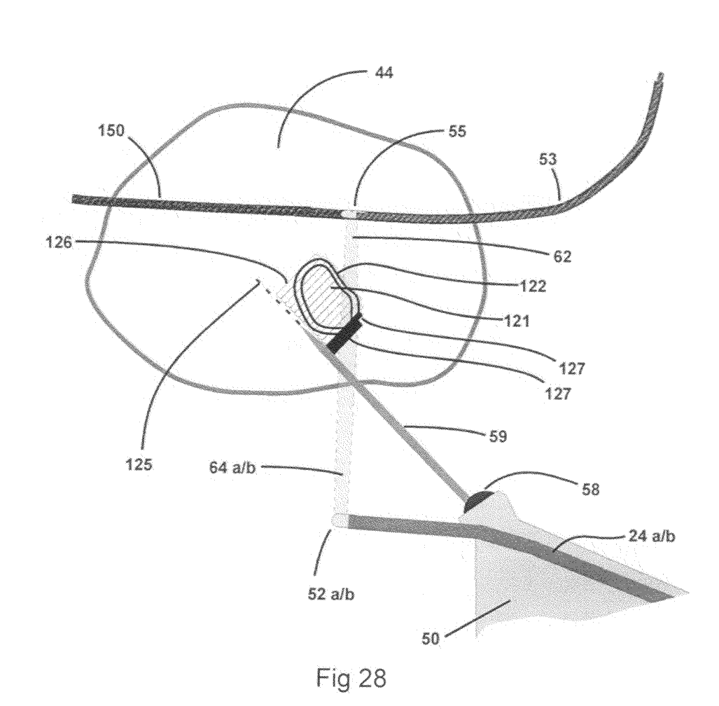

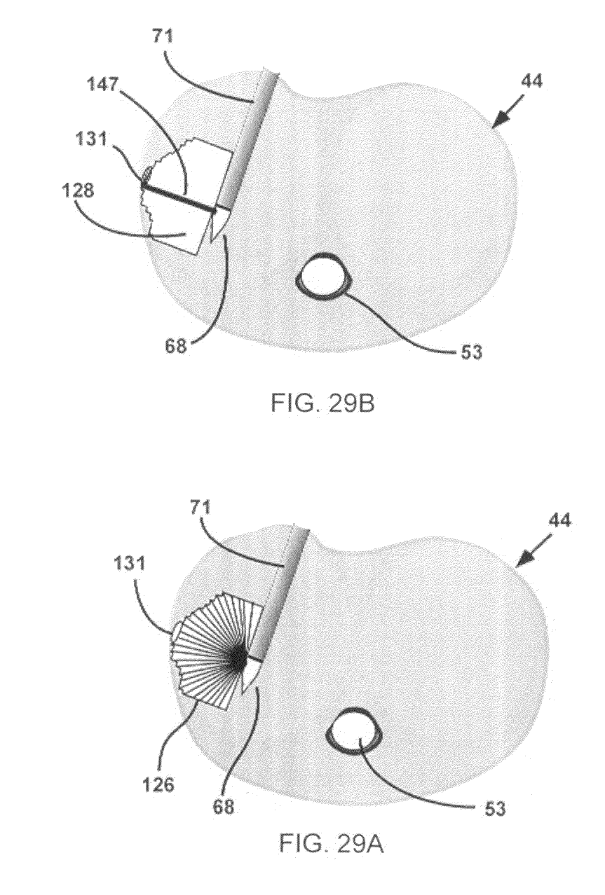

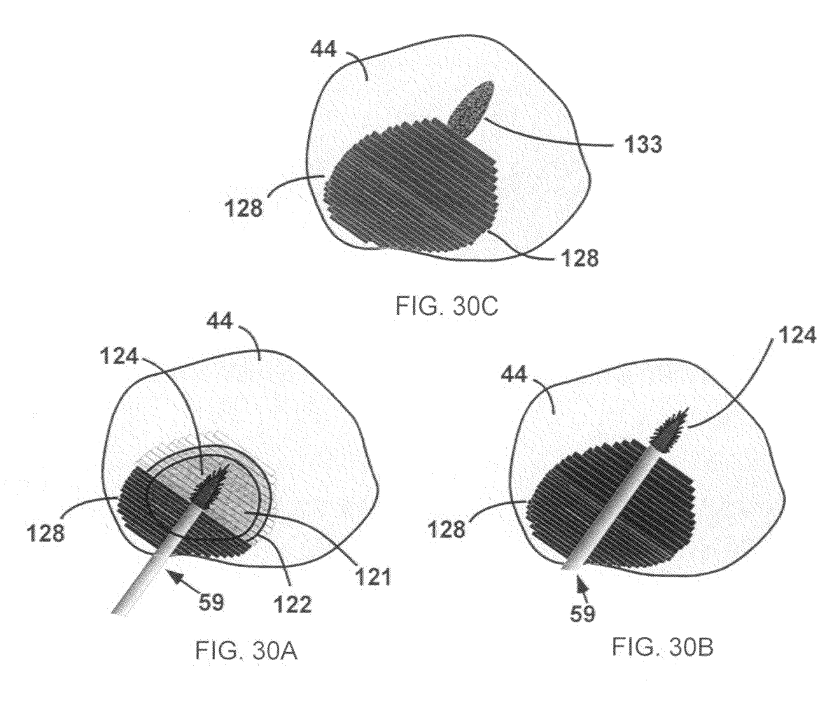

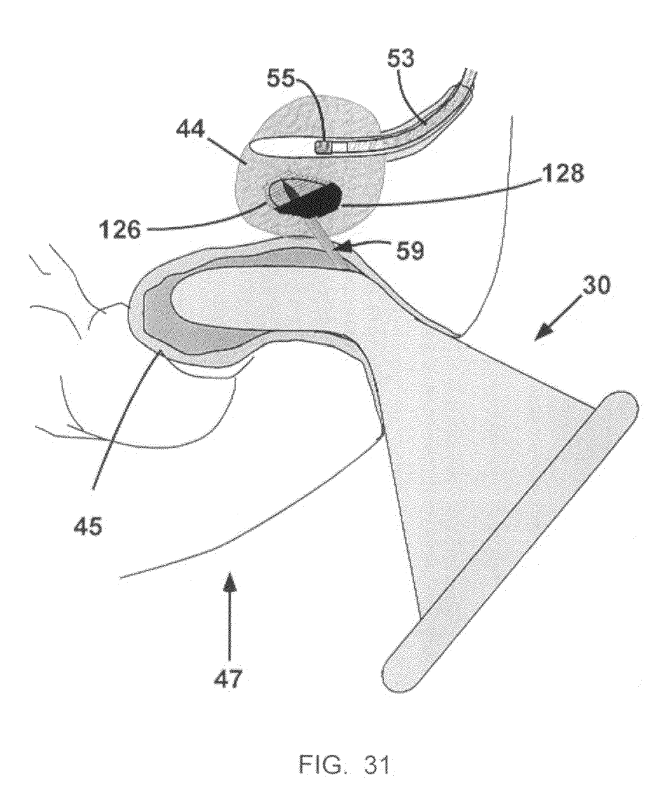

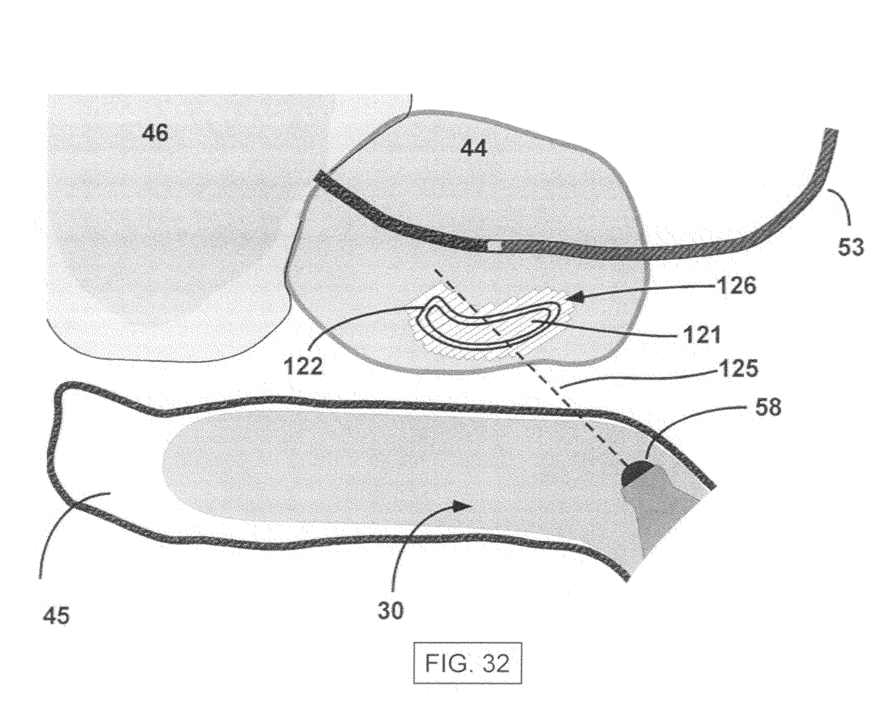

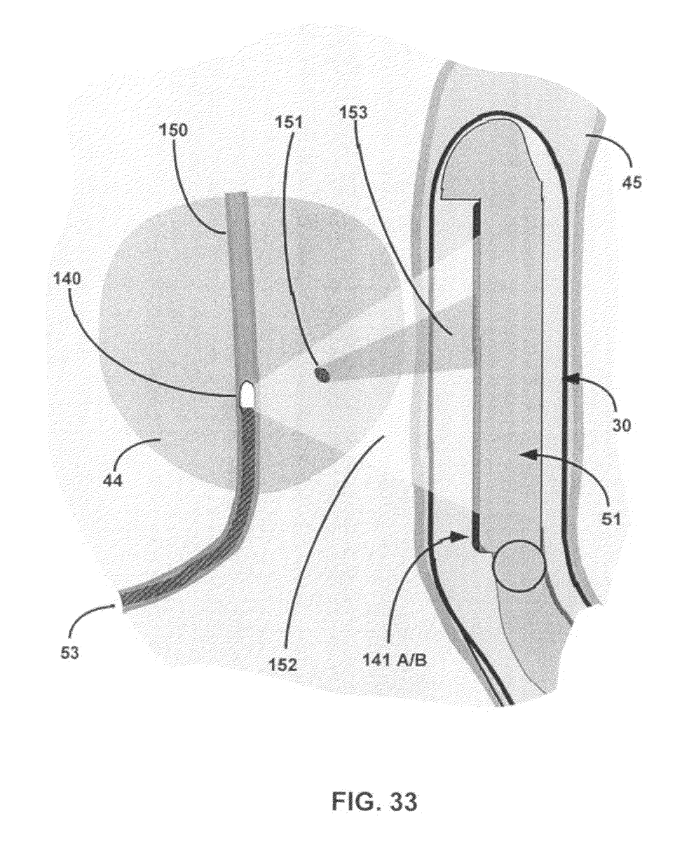

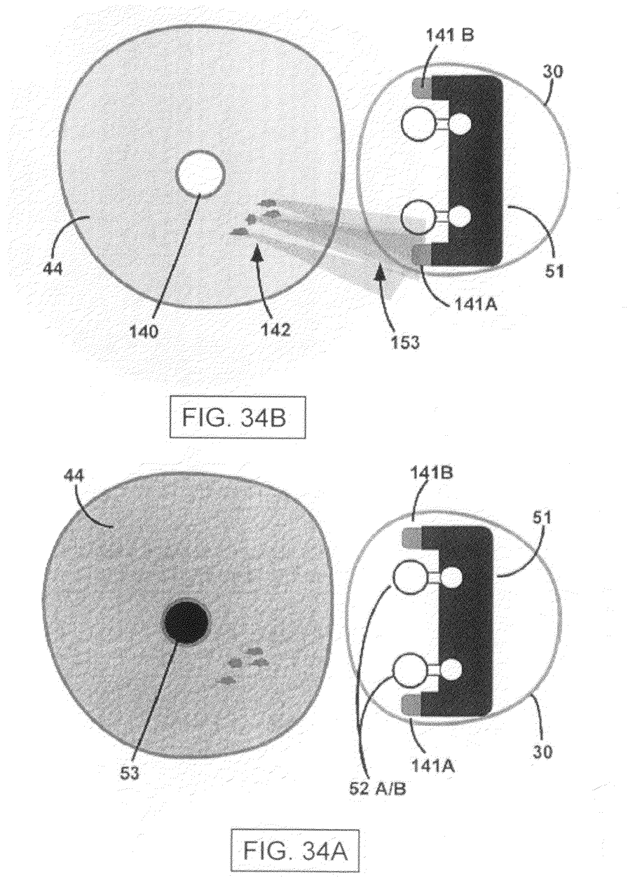

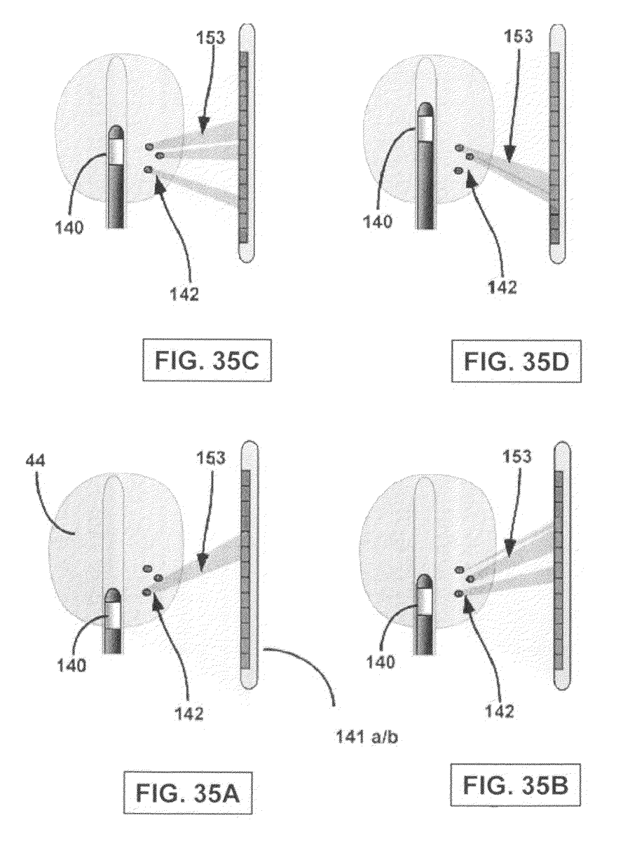

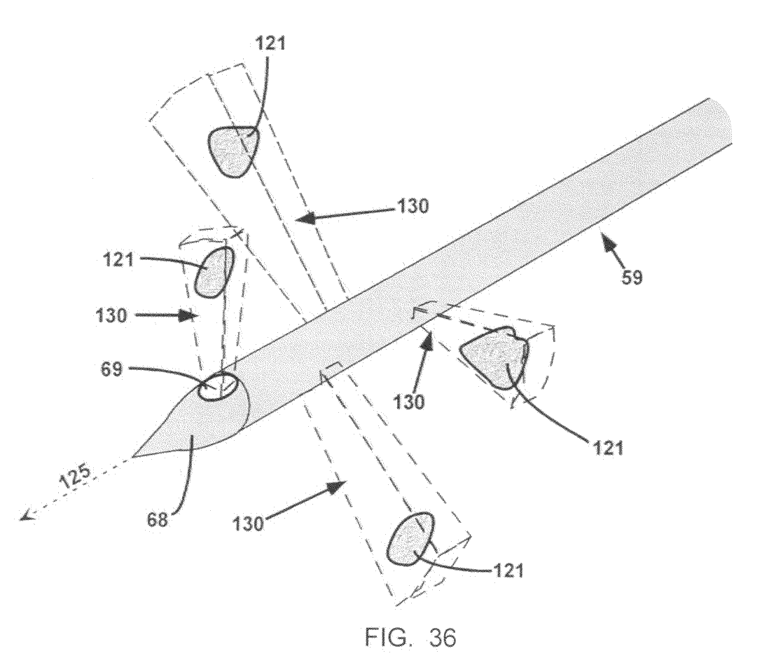

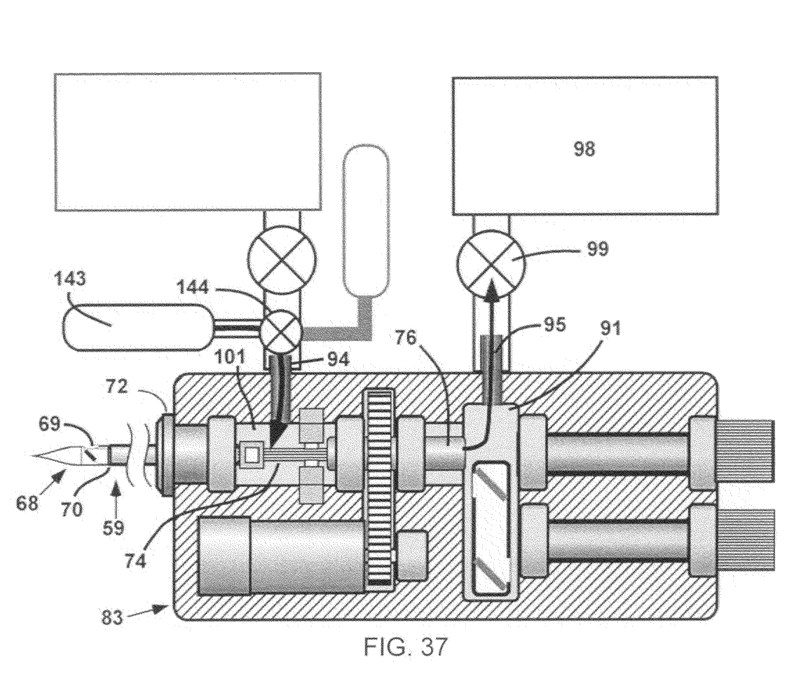

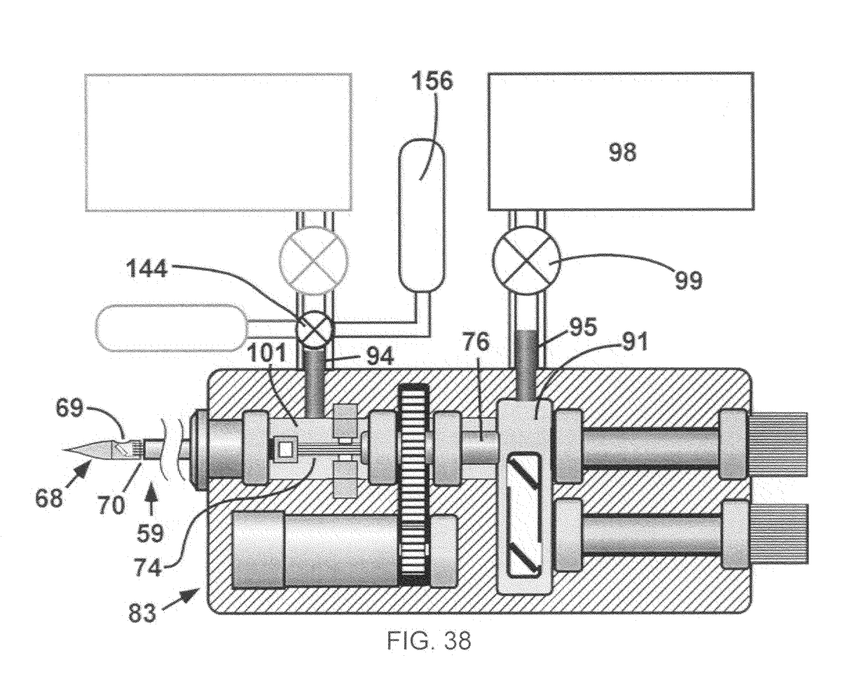

Section 1—An overview of the procedure, display and control systems to place the Transrectal Laser Ablation Probe into the rectum at the desired location to permit the Laser Ablation Needle Applicator to properly perform the eradication process, utilizing technologies previously disclosed in the MedSci System for prostate diagnosis (U.S. Pat. No. 6,824,516). Drawings associated with this section are: FIGS. 1A/B/C FIG. 2, FIG. 3, FIG. 4, FIG. 5, FIG. 6, FIG. 7, FIG. 8, FIG. 9, FIG. 10, FIG. 11, and FIG. 12.
Section 2—Description of the Components that constitute the Laser Ablation Subsystem and how they interact to accomplish the desired total eradication of the mapped tumor with an absolute minimum of collateral damage. Drawings associated with this section are: FIG. 13, FIG. 14, FIG. 15, FIG. 16, FIG. 17, FIG. 18, FIG. 19, FIGS. 20 A/B/C/D/E, FIGS. 21 A/B/C, FIG. 22, FIG. 28, and FIGS. 29 A/B.
Section 3—A detailed description of the incorporated mechanisms whereby the Physician can inspect the interior of the created cavity to verify complete removal of malignant tissue, after the ablation procedure is complete. Drawings associated with this section are: FIG. 22 and FIGS. 29 A/B.
Section 4—A detailed description of the functions used to monitor the actions of the Transrectal Laser Ablation subsystem, which provides for robotic assistance for the treatment process. Drawings associated with this section are: FIG. 10, FIG. 12, FIG. 27 and FIG. 28.
Section 5—A detailed description of the ablation pattern techniques used for tumors of different sizes, locations, and shapes (including technology addressing cell dislodgment). Drawings associated with this section are: FIGS. 23 A/B, FIGS. 24 A/B/C/D, FIG. 25, FIGS. 26 A/B, FIGS. 30 A/B/C, FIG. 31, FIG. 32 and FIG. 36.
Section 6—A detailed description of the Optical System Augmentation Embodiment. Drawings associated with this section are: FIG. 33, FIGS. 34A and 34 B and FIGS. 35A/B/C/D.
Section 7—A detailed description of the mechanisms providing support closure of the created cavity. Drawings associated with this section are: FIG. 37 and FIG. 38
(Note: This procedure is not different than that described in U.S. Pat. No. 6,824,516, but the routing of the functions through the Command and Routing Cassette 83 are different, so are shown for continuity and clarity of the description.)

### Section 1

An overview of the procedure, display and control systems to place the Transrectal Laser Ablation Probe into the rectum at the desired location to permit the Laser Ablation Needle Applicator to properly perform the eradication process, utilizing technologies previously disclosed in the MedSci System for Prostate diagnosis (U.S. Pat. No. 6,824,516). Drawings associated with this section are: FIGS. 1A/B/C, FIG. 2, FIG. 3, FIG. 4, FIG. 5, FIG. 6, FIG. 7, FIG. 8, FIG. 9, FIG. 10, FIG. 11 and FIG. 12.

FIG. 1A, is a schematic showing a side view of the patient chair with the major elements: The fixed base 2, the vertical movement 11 the angle adjustment 6, the slideable backrest 5, the leg rests 4 and the elastography element belt 3, which is attached to hip fences 8.

FIG. 1B is a top view of said chair 1 showing leg rests 4, back rest 5, hip fences 8, and elastography belt 3.

FIG. 1C is a cross sectional view of said chair 1, showing elastography belt 3 removably attached to hip fences 8.

FIG. 2 is a side view of the major elements of the configuration of the disclosed system. Chair 1 on fixed base 2 is shown in proper geometric relationship to electronics tower 13, which supports transurethral system elements 16 and 17, as well as display screen 15 and touch control panel 14. Transrectal laser ablation subsystem moveable base 10 is also shown in the correct position for the procedure. Transrectal laser ablation subsystem 9 is shown at the start position, down and away from the patient chair 1.

The movement of the transrectal ablation laser subsystem 9 and thus of transrectal probe 30 is controlled by joystick 12 under guidance by the Physician.

FIG. 3 is a perspective view showing the interchangeability of the moveable base 7 of the prostate cancer detection and mapping subsystem of prior U.S. Pat. No. 6,824,516 and the moveable base 10 of the herein disclosed transrectal laser ablation subsystem 9 (shown in FIGS. 4 and 3), into the identical position with respect to chair 1 and electronic tower 13. In both cases the moveable base 7 or 10 is locked into place via interlocks 18.

FIG. 4 is a side view showing the transrectal laser ablation subsystem 9 rocked to the correct angle for entry of the transrectal probe 30, into the patient anus. The transrectal laser ablation subsystem 9 is shown moving forward on base 10 as the bellows cover 19 distorts to accommodate the rocking action. Forward interlock 18, between base 10 and chair base 2, is shown in correct relationship.

FIG. 5 is a side view of the attachment point of transrectal probe 30 to transrectal subsystem 9. To assist the Physician in the placement of the Transrectal probe, the system provides real time optical and pressure data. Three video cameras are incorporated into the Laser Ablation subsystem 9. Camera 31 is mounted above and behind transrectal probe 30, on the upper surface of the laser ablation subsystem 9, giving a perspective of the perineal area and anus from above. A second camera 32 is mounted ahead of and below the transrectal probe 30 for the lower perspective of the perineal area and anus. The tip of the transrectal probe houses a forward-looking camera 35, such that it will show the passage through the anus and the interior of the rectum from that vantage point. Above and below the probe tip camera 35, are pressure sensors 33 and 34, which will contact the walls of the anus during insertion, providing pressure data, which is displayed for the Physician.

FIG. 6 shows the outputs of the sensors identified in FIG. 5, which are displayed on display screen 15 (shown in FIG. 2), which is mounted at eye level on electronics tower 13. These displays combine to let the Physician control the movement of the Transrectal probe 30 through the anus and into the rectum while staying in the center of the passage, thus providing minimal off-center distortion of the anus and less discomfort for the patient. The on-screen display consists of the following elements. At the top is the video display 39 from the upper camera 31. In the middle is the display 42 from the transrectal probe nose camera 35. To the right of display 42 is a vertical stack of two digital displays. The upper right digital display 38 shows the reference angle used by the Physician to direct the transrectal probe 30 through the anus and into the rectum as recorded during the original diagnostic procedure using the detection and mapping probe. The lower digital display 37 shows the current angle of the transrectal probe. To the left of center are two more digital displays. The upper of these digital displays 41, shows the output from upper transrectal probe pressure transducer 33. The lower of these two displays 40, shows the output of lower transrectal probe pressure transducer 34. Below these displays is located the video display 36 from the lower camera 32.

FIG. 7 is a sectional, anatomical schematic, side view, showing the tip of transrectal probe 30 at the correct angle to move forward and up through the patient rectum. The pressure sensors 33 and 34 engage the anus as the Physician moves the tip of the Transrectal probe 30 in the XYZ coordinates by applying the appropriate pressure to the joystick 12. The angle of attack is likewise adjusted by control inputs from the Physician, who brings the Transrectal probe 30 forward and up to a point contacting the anus 43, with full visibility of all motion in real time via the 3 cameras. The appropriate movement through the anus 43 is to rock the probe to a steeper angle as it passes through the anus 43 and then back to a flatter angle as it is positioned within the rectum 45. The on-screen guidance will show the angle of the previous insertion, screen display 38. The pressure transducers 33 and 34 serve as a check for this part of the procedure. As the transrectal probe 30 is inserted into the rectum 45, the pressure readouts should be kept the same. This verifies that the probe is centered in the passageway. All real time and historical data appears on display screen 15 as shown in FIG. 6. (NOTE: the movement is described relative to transrectal probe 30, however to achieve that movement the entire laser ablation subsystem 9, to which said transrectal probe is mounted, moves.)

FIG. 8 shows that after proper placement of the transrectal probe with its tip camera 35 and associated illuminator 35a into the patient's rectum 45, the video input provides for the Physician a view of the upper part of the rectum and lower colon. The transrectal probe is now in the proper relationship to prostate 44 and urinary bladder 46.

FIG. 9 shows that when the Transrectal probe is in place, the Physician initiates a water fill of the rectum utilizing the touch screen interface 14. This is done to provide an acoustic pathway for dual Ultrasound Scanners 52 a/b (shown in FIG. 10 and FIG. 11) within Transrectal probe 30, which will monitor the procedure along with the transurethral ultrasound scanner 55. Water is injected into the rectum via port 48 on the lower portion of the transrectal probe 30. The amount of water necessary to fill the rectum is known from the previous diagnostic procedure. This does not differ from U.S. Pat. No. 6,824,516.

FIG. 10 illustrates an additional element of spatial data for control, the base MedSci system U.S. Pat. No. 6,824,516 incorporates a magnetic position sensing system that tracks the position and 3-D relationship of the endoscopic components of the system. Both the transurethral and transrectal Ultrasound scanners and the Transrectal probe body carry magnetic sensors, 54a/b and 56 respectively, which provide their positions in 3-D space and their relationship to one another. This information is tracked in real time and all data is supplied to the controlling computer. All tracking information is provided on screen 15 for the Physician, in a sectional, schematic, anatomical view showing both the transrectal probe 30 in situ within the rectum 45, and the transurethral catheter probe 53 in situ within the prostatic urethra 150, within lower abdomen 47. Within transrectal probe 30 the inner cone 50 supports the transrectal probe backbone 51. The transrectal ultrasound scanner elements 52A/B, together with their respective magnetic markers 54 A/B are slidably mounted to backbone 51. They are connected to transrectal ultrasound scanner drive mechanism 24A/B by transrectal ultrasound scanner drive cables 25A/B, which serve both as the signal connection and to transfer the movements of transrectal ultrasound scanner drive mechanism 24A/B to the transrectal ultrasound scanner elements 52A/B.

The transurethral ultrasound scan element and connected magnetic marker 56 are slidably placed within transrectal catheter probe 53 and moved through prostatic urethra 150 within prostate 44 by the transurethral subsystem mechanical movement 16, which is mounted on electronics tower 13. This does not differ from the prior U.S. Pat. No. 6,824,516.

FIG. 11 is a top view of the transrectal ultrasound probe 30 showing the arrangement of the dual ultrasound scanner elements 52A/B from the transrectal ultrasound scanner drive mechanism 24A/B which are connected to and driven by linear movement 154, through transrectal ultrasound scanner drive cables 25A/B, which are slidably attached to the upper surface of inner cone 50, within transrectal probe cover 60, passing on either side of needle pivot 58 into transrectal probe 30. The tip of laser ablation needle applicator 59 is shown emerging from needle pivot 58.

FIG. 12 is a cross-sectional view of a given step n, of the scanning process, showing the individual scan patterns 64A and 64B of transrectal ultrasound scanning elements 52A and 52B which are shown slidably attached to transrectal probe backbone 51 by transrectal ultrasound scanner movement guides 61A and 61B within transrectal probe cover 60. The transurethral probe/catheter is shown in place within the prostatic urethra within the prostate 44, together with its ultrasound scan pattern 62. The overlap area of the three ultrasound scan patterns is designated as 148 and overlays the largest volume of prostate 44 to provide the best detection of tumors. This does not differ from the prior U.S. Pat. No. 6,824,516.

### Section 2

A description of the Components that constitute the Laser Ablation Subsystem and how they interact to accomplish the desired total eradication of the mapped tumor with an absolute minimum of collateral damage. Drawings associated with this section are:

FIG. 13, FIG. 14, FIG. 15, FIG. 16, FIG. 17, FIG. 18, FIG. 19, FIGS. 20 A/B/C/D/E, FIGS. 21 A/B/C, FIG. 22, FIG. 28, and FIGS. 29A/B.

FIG. 13 is a sectional side view of the laser ablation subsystem 9, which contains the elements of the present invention. The laser ablation subsystem is movably mounted to moveable base 10. It has three degrees of freedom: towards or away from the patient chair 1, up and down, and rocking in the vertical plane. The first two movements do not differ from the prior U.S. Pat. No. 6,824,516. The vertical plane rocking is provided by two pairs of jacking elements 20A/B at the front, and 21 A/B at the back. All movements are controlled by the Physician via joystick 12. As the Transrectal probe is maneuvered into position, the movement will be slowed down from the original speed with which the probe was moved up to the anus 43. The Physician selects speed of movement of the probe relative to the control pressure input from the joystick via a control button 145 on the joystick 12. Initially the movement is relatively quick and then as the probe tip approaches the anus 43, to increase the control sensitivity, the Physician selects the second speed range, in which the probe movement speed in response to the control pressure is half of the original. The Physician has 4 speed ranges available and a thumb button 146 on the joystick controls the slowest speed, such that the pressure on the joystick 12 controls the direction of movement, but the actual movement is stepped by clicking said thumb button 145. One click equals one millimeter of movement or one degree of rotation depending on the control input.

Laser ablation subsystem 9 houses the nexus element of the present invention, the Control and Routing Cassette 83, which provides most of the functionality of the invention. Control and Routing Cassette 83 is held and moved by a series of mechanical movements.

These mechanical movements differ only in detail from those described in prior U.S. Pat. No. 6,824,516. They consist of: extensional movement 29, which holds control and routing cassette 83. The angle of that movement is controlled by vector movement 28, which is in turn moved rotationally by azimuthal movement 27. Said azimuthal movement is held at a neutral angle (relative to the patient in chair 1) by semi-circular laser movement support bracket 26. The shape of the laser movement support bracket 26 (see also FIG. 14) provides room for the fiber optic cables 66 and 67, which respectively connect the ablation laser generator 23 and the fluorescence verification system 22 to the control and routing cassette 83. This permits said fiber optic cables to follow the movements of said cassette as it moves the laser ablation needle applicator 59 through the procedure. To facilitate the freedom of this movement, fiber optic connectors 88 a/b are attached to rotating tube optical pathways 93 a/b, within the control and routing cassette 83. Optical pathways 93 a/b have rotary bearings 87 at each end as shown in FIG. 17, FIG. 18 and FIG. 22 to facilitate the freedom of movement of fiber optic cables 66 and 67.

FIG. 14 shows a schematic view of the frontal aspect of the stacked mechanical movements of the laser ablation subsystem. Moveable base 10 supports jacking elements 20A/B and 21 A/B within bellows cover 19. Semicircular laser movement support bracket 16 supports azimuthal movement 27, which supports vector movement 28, which supports extensional movement 29, which supports control and routing cassette 83. At the top is inner support cone 50, which is mounted to the outer shell of laser ablation subsystem 9 and does not move, relative to said subsystem. Needle pivot 58 is at the top of inner support cone 50. Other than being larger, to accommodate the additional features of the present invention, the arrangement does not differ from the prior U.S. Pat. No. 6,824,516.

FIG. 15 is a side view of the laser ablation needle applicator 59 with the enclosed rotating tip drive shaft 82. Starting at the proximal end, drive shaft 82 is a non-conductive assembly. The base 76 is thickened to support drive gear 75 and contains a central-axial lumen 81 (FIG. 16), which is open at both ends of drive shaft 82. The thickened area of drive shaft 82 continues forward of drive gear 75 is aft shoulder 73 which will interface to a rotary bearing (See FIG. 17). Forward of shoulder 73, the diameter of drive shaft 82 is reduced for an exposed, grooved region 74, which serves a number of functions illustrated in subsequent drawings. Moving forward, the grooved region 74 of drive shaft 82, together with central-axial lumen 81 extends inside the full, length of the laser ablation needle applicator 59. The drive shaft 82 passes through mounting boss 72 and forward of that, non-rotating shell 71. Grooved region 74 of driveshaft 82 protrudes slightly from the distal end of non-rotating shell 71. This protruding tip is bonded into rotating tip 68 as will be described in subsequent drawings. A short length of grooved region 74 remains exposed through annular slot 70, between rotating tip 68 and non-rotating shell 71. A forward shoulder 77 is added to drive shaft 82, to interface with a rotary bearing 87, just behind mounting boss 72 as will be seen in subsequent drawings. This shoulder encloses a portion of grooved area 74 but does not occlude the grooves.

FIG. 16 is a perspective, schematic of the forward end of the laser ablation needle applicator 59. The drive shaft 82 is shown within non-rotating shell 71. The central-axial lumen 81 is shown with the laser ablation beam emerging into central cavity 80 of rotating tip 68, striking 45-degree mirror 78, which is mounted on post 79, such that it rotates with the rotating tip 68 which is bonded to drive shaft 82. The laser ablation beam 147 is deflected at 90 degrees off axis and exits the rotating tip 68 through needle side port 69. Needle side port 69 is in fixed relationship with the 45-degree mirror 78, thus as tip 68 is rotated by drive shaft 82, the emerging Holmium laser beam 147 is swept across the face of tumor tissue adjacent to said port and movement. The laser ablation needle applicator assembly 59 with drive shaft 82 is mounted on control and routing cassette 83, which acts as the nexus of all of the support functions for the laser ablation process.

The control of the application of the Holmium laser beam 147 to the detected tumor lies with the design of the laser ablation needle applicator 59 and the control and routing cassette 83. The control and routing cassette 83 and therefore the attached laser ablation needle applicator 59, is moved by a combination of mechanical movements.

FIG. 17 is a sectional, schematic view of Control and Routing Cassette 83. Said cassette splits into two vertical halves, such that the internal shape of the internal cavities holds all components in the correct relationships. All necessary lumens and ports are integrated into the design. Starting at the aft end of said cassette, two shaped lumens, vertically arranged, hold the following components: rotating tube optical pathways 93A and 93B are each fitted with a sealed bearing 87 at each end, the aft end of each of the rotating tube optical pathways carries a fiber optic connector, fiber optic connector 88A is attached to tube pathway 93A, and fiber optic connector 88B attaches to tube pathway 93B. This arrangement maintains optical alignment by allowing the connectors to swivel as the attached fiber optic cables 66 and 67 follow the movements of the control and routing cassette 83 during an ablation procedure. The forward end of each of the rotating tube optical pathways is open into optical chamber 91. The upper pathway 93A is inline with the central-axial lumen of the tip drive shaft 82. The lower pathway 93B is inline with the lower port of the optical switch 90, when said switch is in the upper or fluorescence verification position (FIG. 22). Other functional elements incorporated into the control and routing cassette 83, which will be detailed in subsequent drawings are: optical switch 90, which is mounted on optical switch position mechanism 92, vacuum port 95, which enters optical chamber 91, forward chamber 101 with forward port 94, and a split commutator (see FIG. 20) located in forward chamber 101.

FIG. 18 illustrates that the ablating Holmium laser beam 147 is produced by ablation laser generator 23. The beam exits said generator through fiber optic connector 88C, passes through fiber optic cable 66 and fiber optic connector 88A into rotating tube optical pathway 93A. The laser beam emerges from said rotating tube optical pathway into optical chamber 91. The beam then exits that chamber, passing into the central-axial lumen 81 of the tip drive shaft 82. The beam passes entirely through said central-axial lumen, emerging into the central cavity 80 of the rotating laser ablation needle tip 68 where it is deflected 90 degrees off axis and exits rotating tip 68 through needle side port 69. Rotating tip 68 is turned by drive shaft 82, which is fixed to said tip. The rotary motion is provided by rotary drive gear 75, which is driven by motor drive gear 86. Both of these gears are housed in drive chamber 84 of control and routing cassette 83. Motor drive gear 86 is connected to variable speed/variable direction rotary drive motor/encoder 85 and activated under the direction of the Physician, mediated through the control computer. All rotating elements are supported by a series of sealed rotary bearings 87.

FIG. 19 shows that as the tissue is ablated by the action of the laser beam, it is necessary to remove the by-product vapors produced by the tissue erosion process. These vapors could attenuate the laser beam and they need to be cleared from the cavity as it is being created. This is accomplished via the vacuum system 98, which is located external to the control and routing cassette 83 but within the laser ablation subsystem 9. Vacuum is applied to the cavity through needle side port 69, so that the vapors pass through the central axial lumen 81 of driveshaft 82. The vapors are drawn out of the base of the tip drive shaft 76 into optical chamber 91. The vapors are drawn out of optical chamber 91 through vacuum port 95 to the vacuum system 98. Vacuum modulation valve 99 provides the means for the Physician to control this extraction for best performance. This action clears the vapors created by the ablation process from the optical pathway to prevent interference with the Holmium laser ablation process.

To dilute the vapors being produced within a created cavity 128 (see FIG. 31), inert gas is injected into said cavity through annular slot 70. This also provides the ability to control the pressure within said cavity, replacing the ablated volume being extracted by the vacuum system 98 and holding the cavity open. Said annular slot exposes a series of axial grooves 106 a/b/c/d, which are part of the grooved portion 74 of drive shaft 82 (see FIG. 15 and FIG. 20D) in the surface of tip drive shaft 82. These slots extend along the drive shaft into the forward chamber 101 and are open to the interior of that chamber. Chamber 101 is connected to inert gas system 96 via forward port 94. Gas modulating valve 97 is used to control the flow of inert gas through the described pathway and into the created cavity. It should be understood that these two opposing actions would need to be balanced. However, it is anticipated that, using the available controls, a technique will evolve quickly with testing. This available pathway serves other functions which will be described later, but the primary function is to inject inert gas into the created cavity 128 from inert gas system 96 at a modulatable rate and pressure to both flush vapors created by the vaporization out of the optical pathway of the Holmium laser beam 147 and to hold said created cavity open and accessible during the ablation procedure. Gas flow is controlled by gas modulation valve 97. (Note: This action can be continuous or pulsed to create the most effective action for efficient removal of the vapors)

FIGS. 20A/B/C/D/E are sectional, schematics illustrating the layout of the split commutator assembly and the functionality of the various elements of drive shaft 82.

The function of the commutator is to supply power to the tip heater 109 (FIG. 21 B/C)
FIG. 20A is a side view of forward chamber 101 of control and routing cassette 83 showing drive shaft 82 entering said chamber through aft shoulder 73, and rotary bearing 87. Within forward chamber 101 is the grooved, exposed portion 74 of said drive shaft. At the proximal end of said forward chamber, is located one pair of commutator brushes 102 a/b, which are disposed by 180 degrees and bear against paired driveshaft conductor segments 104 A/B, which run axially forward in slots in the surface of the drive shaft 82. These are likewise disposed by 180 degrees on the drive shaft. These elements are of one polarity. At the distal end of forward chamber 101 is the other half of the commutator. Paired commutator brushes 103 A/B are disposed by 180 degrees and this assembly is 90 degrees rotated from paired commutator brushes 102 A/B. Commutator brushes 103 a/b bear against paired driveshaft conductor segments 105 A/B, which run axially forward in slots in the surface of the drive shaft 82. These are likewise disposed by 180 degrees on the drive shaft. This second group of elements is of the opposite polarity as the first described group of elements. Segment pair 105 A/B is at 90 degrees to segment pair 104 A/B. On exiting forward chamber 101 the named elements of the forward portion of drive shaft 82 pass through forward shoulder 77, a second rotary bearing 87, the needle mounting boss 72 and the non-rotating needle shell 71. Forward chamber 101 also interfaces to the exterior of cassette 83 via forward port 94, which has multiple functions, which will be detailed in subsequent drawings.
FIG. 20B is a top view of these same components.
FIG. 20C is a cross-sectional, schematic view of the split commutator brushes, showing the geometrical relationship of both paired brush sets and to the drive shaft 82.
FIG. 20D is a cross-sectional, schematic view of the arrangement of the identified conductor segment pairs 104A/B and 105A/B as they are located on the grooved surface 74 of drive shaft 82. It also shows a cross-sectional view through non-rotating needle shell 71 and grooved surface 74 of drive shaft 82 showing how grooves 106A/B/C/D together with non-rotating shell 71 form a series of passageways through which inert gas or other materials, introduced through forward port 94 into forward chamber 101 can be forced to flow forward along the outside of the rotating drive shaft 82, while the Holmium laser beam and the extracted vapors pass through the central-axial lumen 81 of said drive shaft.
FIG. 20 E Illustration showing inert gas exiting grooved drive shaft through annular slot 70.

FIG. 21A is a perspective view of the forward end of drive shaft 82 showing central-axial lumen 81, both conductor segment pairs 104A/B and 105A/B, and the four passageway grooves 106A/B/C/D in the correct geometric relationship.

FIG. 21B is a sectional view of the geometry and attachment of rotating needle tip 68 and the supporting elements: the rotating tip drive shaft 82 with conductor segments 104A/B and 105A/B, the rotating tip mating conductors 107A/B and 108A/B, and their connection to tip heater 109. Also shown is annular slot 70 and the area where drive shaft 82 is bonded to rotating tip 68, which is also the area where electrical connection is made between the drive shaft paired conductive segments and the paired tip conductors to complete the circuit.

FIG. 21C is a sectional, schematic view of the electrical connections within rotating tip 68 between the tip heater 109 and the drive shaft conductor segments.

FIG. 22 shows the control and routing cassette 83, routing of the fluorescence illuminating energy and return to detector 113 (see Section 3 for detail description).

FIG. 28 shows capacity for real-time imaging and is discussed in Section 4.

FIGS. 29 A/B show delivery of optical energy for fluorescence inspection and is discussed in Section 3.

### Section 3

A detailed description of the incorporated mechanisms whereby the Physician can inspect the interior of the created cavity to verify complete removal of malignant tissue, after the ablation procedure is complete. Drawings associated with this section are: FIG. 22 and FIGS. 29A/B

FIG. 22 There are two methods of treatment verification available: The primary indication of treatment effectiveness is the overlay of the ultrasonic image outline of the created cavity 128 over the outline of the mapped tumor 121 with margins 122 to show that the cavity has replaced the entire volume of tissue which had contained the tumor (see FIGS. 28 and 31).

The secondary indicator, Fluorescence Examination, is detailed here: this Fluorescence Verification process takes advantage of the fact that malignant tissue is known to fluoresce with a specific response when illuminated at the appropriate wavelength of light. This is accomplished at the direction of the Physician. FIG. 22 is a sectional schematic of the operation of the fluorescence verification system 22. Illumination having the proper spectral content is produced in fluorescence generator 112. The optical signal passes through signal splitter 111 and exits system 22 via optical fiber connector 88D and fiber optic cable 67. The optical signal enters the control and routing cassette 83 through fiber optic connector 88B and rotating tube passage 93B. The optical signal enters optical chamber 91 and enters the lower port of optical switch 90, which has been moved to the verification (upper) position within chamber 91 by optical switch movement mechanism 92, where it is deflected by 45-degree mirror 90b, then deflected again by 45-degree mirror 90a. The illumination exits the optical switch 90 and enters the axial central lumen 81 of the drive shaft 82. The optical signal then follows the same route as the Ablation Laser Beam 147, which is turned off for the verification procedure. On arriving at the 45-degree minor 78 in the rotating tip 68, it is directed out through needle port 69 to illuminate the interior of the created cavity 128. The tip of the laser ablation applicator needle 59 is incrementally moved through the created cavity 128. At each increment the rotating tip 68 moves through a 360-degree rotation. Any malignant tissue remaining 131 will fluoresce with a characteristic spectral signature. That reflected signal passes back through the same pathway to the signal splitter 111 where it will deflect into the fluorescence detector 113. If residual malignant tissue is detected, it will be displayed on data display 15 for the Physician. The optical switch 90 can be moved to the lower position, the ablation laser generator 23 reactivated, and the detected residual tissue can be further ablated.

FIG. 29A is a sectional, anatomical schematic that shows the application of the laser fluorescence to the interior of a created cavity 128 in tumor 44 via the needle side port 69 of laser ablation needle applicator 59 as described in FIG. 22, with residual malignant tissue 131 being illuminated, which will return a signal to detector 113.

FIG. 29B is a sectional, anatomical schematic that shows the re-application of the Holmium laser beam 147 to the interior of created cavity 128 in tumor 44 via the needle side port 69 of laser ablation needle applicator 59 as described in FIG. 18, with residual malignant tissue 131 being targeted for eradication. This process can be invoked by the Physician as needed.

### Section 4

A detailed description of the functions used to monitor the actions of the Transrectal Laser Ablation subsystem, which provides for robotic assistance for the treatment process. Drawings associated with this section are: FIG. 10, FIG. 12, FIG. 19, FIG. 27, and FIG. 28.

FIG. 10 is described in Section 1 and supports Section 4.

FIG. 12 is described in Section 1 and supports Section 4.

FIG. 19 is described in Section 2 and supports Section 4.

FIG. 27 is a sectional, anatomical schematic illustrating the overall process for monitoring and control of thermal treatment operations. This does not differ from the approach used in prior U.S. Pat. No. 6,824,516. At the beginning, the first step will be to map again in real time the prostate location and cancer area to be treated in relationship to the location of the treatment subsystem, utilizing the transurethral and transrectal ultrasonic imaging systems. Having acquired new real-time imaging and compared the screen display of the historical and current images of the cancer, a computer-generated 3-D treatment grid is produced of the tissue volume containing the tumor and the planned treatment safety margins. This will facilitate control of the treatment process. The time for completion of each eradicating sweep is a function of the selected constant speed rate and the angular distance between the Laser Applicator Needle 59 and the wall of the cavity to be created. Also, the depth of the Holmium laser penetration has been premeasured for various rotational speeds for the needle applicator (i.e. time on target for the laser) thus the computer software can keep track of the tissue volume eradicated vs. planned volume by counting sweeps. Such information, in conjunction with the known spacing of the computer-generated mapping grid, can be utilized by the software to provide guidance for when and how often to apply verification of treatment status with the laser fluorescence capability. These integrated modalities, together with the real-time ultrasonic imaging of the cavity creation, function to provide precise control over the size, shape and orientation of the tumor eradication process with effectiveness verification.

Elements and functions available to apply to this requirement are:
Computer-generated 3-D grid for planning the laser ablations
Capability to track ablation penetration by count of laser sweeps
Dual ultrasonic scanners within transrectal probe 52 A/B
Transurethral ultrasonic scanner 55
Magnetic sensors 56
Capability for “on demand” laser fluorescence confirmation of progress in elimination of tumor tissue
Capability to have computer to control multiple ultrasonic sweeping of the area to each side of the path of the eradication process

The process control afforded by the system over the disclosed tissue removal process allows the Physician to plan and control the procedure for minimal damage to non-cancerous tissue and structures.

The laser ablation needle tip will penetrate the prostate 44 along the designed pathway 125. The needle will stop when it reaches the designed point at which the Tumor ablation process is to begin as specified by the Physician, who can now make a final assessment of the positioning and pathway before initiating the ablation procedure. The on-screen display 15 will show a newly acquired outline of the mapped tumor 121 as a translucent 3-D image with the designed treatment margins 122 in a second color; the position and radial orientation of the Laser Ablation Needle 59 are also shown. Outlines of the position and relationship of both the Transrectal probe 30 and transurethral probe 53 are likewise shown on the screen. The ultrasound scanners will sweep back and forth across the volume of the tissue in a stepwise fashion, shown as 62 and 64A/B.

FIG. 28 is a sectional, anatomical, schematic illustrating how the present invention permits the physician to observe and confirm the removal of tissue from the designated area in real time, via interaction with a transurethral ultrasound scanner 55 as well as dual Transrectal ultrasound scanners 52. Ultrasound monitoring of the cavity creation action takes advantage of the fact that a cavity is impenetrable to ultrasound at diagnostic frequencies and so is the best reflector possible. Therefore as a cavity segment 127 is created by laser ablation needle applicator 59, from planned track 125, transurethral ultrasound scanner will scan that area 62 while transrectal ultrasound scanners 52A/B will simultaneously scan the same area, 64A/B (See also FIG. 12). The reflected energy from the created cavity segment 127 permits the confirmation and tracking of the procedure. This data is overlaid with the outline of the original mapped tumor 121 and margin 122 and displayed for the Physician.

### Section 5

A detailed description of the ablation pattern techniques used for tumors of different sizes, locations, and shapes. Drawings associated with this section are: FIGS. 23 A/B, FIGS. 24 A/B/C/D, FIG. 25, FIGS. 26A/B, FIG. 27, FIG. 28, FIGS. 29A/B, FIGS. 30A/B/C, FIG. 31, FIG. 32, FIG. 36

FIG. 23A is a sectional, anatomic schematic showing laser ablation needle applicator 59 penetrating prostate 44 on a path which is tangential to mapped tumor 121 with it's enclosing Physician specified margin 122. The laser ablation beam is swept through an arc that will enclose the tumor and margin, creating a cavity segment 127.

FIG. 23B is a sectional, anatomic schematic showing laser ablation needle applicator 59 penetrating prostate 44 on a path which is centroid to mapped tumor 121 with it's enclosing Physician specified margin 122. The laser ablation beam is swept through a full 360 degrees, to create a cavity segment 127 that will enclose the tumor and margin,

FIG. 24A/B/C is a sectional, anatomical schematic illustrating stages in the ablation process. The rotating tip 68 of the Laser Ablation Needle 59 is placed at the appropriate start point for an ablation procedure. The Physician then initiates the ablation procedure. The rotating tip 68 at the first axial step begins to sweep the Holmium laser beam 147 over the surface of the tissue to be ablated. The radial depth of penetration and therefore the shape and size of the ablated volume will be equal to the diameter of the laser beam, the rotational speed, and the number of times it passes over the exposed inner surface of the cavity being created at that radii. Since that factor is completely controllable, the created cavity can be tailored to be congruent to the cross section of the tumor 121 at that axial location plus a Physician designated margin 122. The erosional action is illustrated in this drawing, with 24A being the start of the process and 24d the conclusion. The erosional stages are identified as 129 leading up to the final creation of segmental cavity 128. The rotating tip 68 of laser ablation needle applicator is shown axially. Other procedures using Holmium lasers have documented a tissue removal rate of approximately 1 gram per minute. After a tailored cavity 128 has been created, eradicating one cross sectional segment of a mapped tumor, the Laser Ablation Needle 59 steps forward a distance equal to the axial thickness of that created cavity and begins to ablate the next cross sectional segment. In this fashion, the Laser Ablation Needle creates a stack of cavities, each of which eradicates a successive cross-sectional segment of a mapped tumor, until the entire tumor has been vaporized and the tumor volume has been replaced by a combined cavity stack 128, replacing the volume originally occupied by the tumor and margin.

FIG. 25 is a perspective schematic of a typical, wedge shaped joined cavity stack 128 created by the tangential ablation approach. The laser ablation needle applicator is shown with the Holmium laser beam 147 creating the final segmental cavity 127 to complete the planned eradication of mapped tumor 121 with margin 122.

FIG. 26A is a sectional, anatomical schematic illustration of a small tumor 121 overlaid with the planned ablation pattern 126, as laser ablation needle applicator 59 approaches.

FIG. 26B is a sectional, anatomical schematic illustration of the same small tumor 121 overlaid with the planned ablation pattern 126, showing that the entry of the needle applicator 59 will distort said small tumor, invalidating the planned ablation pattern. This is another reason that the tangential approach to the tumor is preferable.

FIG. 30A/B/C is a series of sectional, anatomical, schematics, illustrating an additional function of the laser ablation needle applicator, as follows. Any surgical technique that penetrates a tumor has the possibility of dislodging malignant cells, which can escape to produce other tumors. The present invention provides mechanisms to minimize or eliminate this problem. This is accomplished in two ways: 1. Where possible a tangential ablation is performed. In this manner, the needle never enters the tumor. Only the Holmium laser beam 147 enters the tumor, providing for complete vaporization of the tumor 121 with the designated margin 122, as illustrated in FIG. 25 and FIG. 28, thus eliminating the possibility of dislodged cells.

Where it is deemed necessary by the Physician to penetrate the tumor with the Laser Ablation Needle Applicator 59 using a centroid approach, because of local conditions. The present invention provides mechanisms to necrotize any dislodged cells immediately. The operation of this mechanism is as follows. The volume of the tissue comprising the body of the tumor 121 with defined margin 122 will be vaporized during the procedure, thus presenting no danger. However, the side port 69 through which the ablating laser beam 147 emerges, is of necessity, behind the penetrating point of the tip and will penetrate the back boundary of the tumor to enable complete vaporization of the body of said tumor. The possibility exists that, as the tip penetrates the back boundary of the tumor, it could dislodge cells and push them ahead and to the side. To prevent this potential problem the rotating tip 68 contains a tip heater element 109 which can produce heating levels in the tissue adjacent to said tip, sufficient to necrotize the volume of tissue surrounding the tip thus necrotizing the volume of tissue that would contain any dislodged tumor cells to prevent their escape. The operation of this mechanism is as follows. As laser ablation needle applicator rotating tip 68 approaches the far boundary of the tumor 121 being ablated, the tip heater 109 (FIG. 21) is energized to destroy any cells that might have been dislodged by it's movement and are being pushed by the needle (FIG. 30A) The heat is left on as the tip penetrates the far boundary (FIG. 30B), through a dwell time after the tip has reached it's furthest extension and is ready to be withdrawn. (FIG. 30C) In this way we prevent the escape of tumor cells that could cause secondary metastases or recurrence. An example of the tip heater necrotizing the surrounding tissue is identified as 132. An example of the necrotized volume left by the withdrawal of the needle after the conclusion of the procedure is identified as 133.

FIG. 31 is a sectional, anatomical, schematic of the disclosed system laser ablation needle applicator ablating segments of a planned ablation 126, with approximately half the tumor already having been replaced by created cavity 128. This illustrates a centroid approach to the tumor at a point just before the energizing of the tip heater 109.

FIG. 32 is a sectional, anatomical, schematic illustrating the adaptation of the created cavity to the shape of a mapped tumor. If the tumor 121 main axis is skewed relative to the optimal tangential path or Centroid path to be taken by the Laser Ablation Needle, each created cavity segment can be skewed by selecting the optimal path 125 and then adjusting the number and angle of each planned segment ablation 126. In this way the overall created cavity can be skewed to the tumor 121 orientation.

The shape, orientation and size of the created cavity are set to eliminate the mapped tumor regardless of shape or size. By creating a “stack” of contiguous cavities, each sized and shaped to the particular segment of the mapped tumor 121 being targeted, when the “stack” is complete, the total tumor with margins is eliminated. There is no issue of achievement of uniform treatment coverage of the diseased tissue, as can be the case with other types of thermal modalities. The tumor tissue is completely eliminated. There is no issue of collateral damage; the size, shape and orientation of the contiguous created cavity are all controllable to sub-millimeter precision, by building on techniques derived from the machine tool industry. Only cancerous tissue, with physician set safety margins, is removed. The disclosed system will give the absolute minimum of collateral damage to non-involved tissues and structures.

FIG. 36 Adaptations included in this augmentation embodiment to the MedSci Laser Ablation Treatment System provide maximum flexibility and capability to eradicate any designated volume of tissue in an afflicted prostate, while not destroying non-affected tissue. The eradicated tissue can be noncontiguous. To apply this process for small tumors or small areas, the MedSci Laser Ablation Needle is used in the standard mode to address each volume sequentially.

### Section 6

A detailed description of the Optical System Augmentation Embodiment. Drawings associated with this section are: FIG. 33, FIGS. 34A/B, and FIGS. 35A/B/C/D.

Recent advances in Optical-Spectroscopy suggest that they may be able to enhance the detection and identification of multi-focal and other difficult-to-resolve tumors. Since the MedSci Detection and Mapping system was designed with the inherent flexibility to make use of new technology when it becomes available, adding this capability gives the system another tool that may be particularly relevant when multifocal confirmation is necessary. The optical absorption spectra of tumors in the near infrared range, differs from non-cancerous tissue at the molecular level. This phenomenon can produce a high contrast optical signature due to differential absorption of the tumor tissue versus normal tissue. The criteria for deployment of this embodiment will be: when cancer has been detected, confirmed and mapped in at least one location within a patient's prostate and there is a question as to possible multiple tumor foci, additional analysis will be performed utilizing light energy technology to augment performance from the ultrasonic imaging capabilities.

This enhancement reinforces the probability of an accurate assessment by corroborating the detection and mapping of the cancer condition via the primary ultrasonic imaging capability of the MedSci system. This supports the physician in making an intelligent decision to provide focus treatment of primary and secondary foci with the laser thermal treatment, or to perform a partial radical, or a complete prostatectomy.

FIG. 33 is a sectional, anatomical, side view schematic, of the use of an optical augmentation for conditions where multifocal tumors are suspected. The optical absorption spectra of tumors in the near infrared range differ from non-cancerous tissue at the molecular level. Since light at these wavelengths is known to penetrate tissue to a depth of up to 10 centimeters, the relatively short distances involved in the prostate procedure will produce a high optical contrast. Thus, by placing a moveable illumination source 140 within the prostatic urethra 150 inside the transurethral catheter probe 53, in place of the transurethral ultrasound scanner, the prostate can be illuminated by source 140. Illumination from the optical emitter 140 penetrates the prostate tissue, impinging on the optical detector rows 141 a/b in the transrectal probe 30. 151 is an example of a small, optically dense tumor that is casting a shadow 153, onto the optical detectors 141A/B.

FIG. 34A is a cross sectional view through the prostate 44 and the transrectal probe 30, showing two rows of optical detectors 141 A/B on either side of the prostate-facing side of Transrectal Probe backbone 51, outboard of ultrasound scanners 52A/B. In this view the transurethral ultrasound scanner is in the transurethral probe/catheter 53.

FIG. 34B is a cross sectional view through the prostate 44 and the transrectal probe 30, showing two rows of optical detectors 141 A/B on either side of the prostate-facing side of Transrectal Probe backbone 51, outboard of ultrasound scanners 52A/B. However, in this view the transurethral ultrasound scanner has been replaced in transurethral probe/catheter 53, by light source 140, which is backlighting a previously unseen group of small tumors 142 causing their shadows 153 to fall on detector row 141A.

FIG. 35A/B/C/D are sectional, anatomical, schematics, illustrating that by moving light source 140 along the length of prostatic urethra 150, while the rows of multiple optical detectors 141 A/B remain in fixed position relative to the prostate 44, the changing angle of cast shadows 153 from a representative small group of tumors 142 will cause the point of impingement of said shadows 153 on optical detectors 141A/B to vary, with a pattern that is reflective of the number, size, and relative location of said small tumors. FIG. 34A shows the light source at the beginning of its travel and the progression is forward through FIG. 35D.

### Section 7

A detailed description of the mechanisms provided support closure of the created cavity. Drawings associated with this section are: FIG. 37 and FIG. 38. (Note: This procedure is not different than that described in U.S. Pat. No. 6,824,516, but the routing of the functions through the Control and Routing Cassette 83 are different, so are shown for continuity and clarity of the description.)

FIG. 37 if the tumor and thus the created cavity are small and the physician deems appropriate, the system can alternatively apply a vacuum via the normal pathway of the vacuum source 98 as described in FIG. 18, to collapse the cavity, vacuum valve 99 is then closed. Rotary valve 144 is then activated to permit the flow of a tissue adhesive (chosen from the family of typically used tissue adhesives) from pressurized tissue adhesive source 156. The pressurized adhesive is forced through the same pathway that was earlier used for inert gas injection as described and illustrated in FIG. 19.

FIG. 38 If the tumor and thus the created cavity are large, the system provides the additional capability of filling the created cavity with a tissue gel, to support healing. This is done by partially evacuating the cavity using the external vacuum system 98. The vacuum valve 99 is then closed. Rotary valve 144 in the inert gas line then closes the pathway to inert gas system 96 while opening a pathway to a source of pressurized liquid gel material 156. Gel material is forced through the same pathway that was earlier used for inert gas injection, as described in FIG. 19.

While the foregoing written description of the invention enables one of ordinary skill to make and use what is considered presently to be the best mode thereof, those skilled in the art, will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as claimed. It will be obvious to those skilled in the art, that the principles and mechanism of the described system, while designed for application to prostate cancer, can be extended to address tumors and other tissues in other parts of the body in a manner that would confer the same functional advantages relative to current technology.

### Alternate Embodiments

Alternate embodiments are envisioned for application to tumors that are large and/or located in other areas of the body where the issues of accessibility dictate a different manner of delivering the laser tumor eradication to the volume of tissue containing a tumor.

In this group, guidance is supplied via a combination of: CT or MRI scan derived positional data to locate and map the tumor area during the initial diagnostic procedure. For the ablation procedure, the Laser Ablation system is mechanically coupled to and supplied with positional data by a modified CT scanner. These inputs are used to select the entry point, angle of attack and depth of insertion to correctly position the laser ablation applicator for the eradication procedure. The actual procedure is then guided by local ultrasound scanning and laser fluorescence systems that are mounted directly on the laser ablation applicator itself. Optical viewing can also be provided. Within this group, depending on the size, location and difficulty of access, the laser ablation applicator can take several forms, as appropriate for the conditions and location of the tumor.

We claim:

1. A system for tumor elimination via creation of a conformal, segmented cavity with Physician defined margins, utilizing laser energy—the cavity volumetrically replaces said tumor.

2. The system of claim 1, that includes integration of previously acquired mapping information for treatment planning, together with real-time comparison of current tumor map, planned volume removal, and monitoring of actual in-process volume removal for treatment tracking and verification.

3. The system of claim 2, wherein said system incorporates computer aided robotic control and mechanical movements such that control of the boundaries, size, position and orientation of the ablated volume has a tolerance of less than a millimeter, thus having a capability to bring all Physicians to the same level of performance.

4. The system of claim 1, wherein the system uses a hollow needle with rotatable tip and support mechanisms to deliver a laser ablation beam precisely on target to eradicate a tissue volume in a well controlled fashion, at the direction of a Physician.

5. The system of claim 4, which includes the ability to insure that there is no possibility of cancer cells escaping to cause secondary metastasis by providing mechanisms such that the rotatable tip can generate heat sufficient to necrotize tissue in the immediate vicinity of said rotatable tip, thus destroying any cancer cells dislodged by said tip penetrating a tumor.

6. The use of a single component that consolidates and locates all of the operational elements needed to enable the functionality of the described Laser Ablation Needle Applicator for the targeting and eradication of tissue volumes, referred to herein as the Control and Routing Cassette.

7. The system of claim 6, wherein the system includes the integration of an inert gas injection and vacuum removal of vapor byproducts of the laser ablation of tissue. Also, the ability to modulate the injection of the inert gas and treatment vapor removal so as to keep the created cavity open at all times to facilitate ease of treatment.

8. The system of claim 1, wherein the system has the ability to apply the laser ablation from a path defined by the Physician. Said path is selected to be either tangential to the tumor or centroid to the tumor, using either a vector sweep or an 360 degree rotation respectively, to create a joined series of cross-sectional slices through the tumor and Physician specified margin.

9. The system of claim 6, wherein the system includes the ability to image the interior of the cavity with laser fluorescence energy through the applicator probe to insure that there is no remaining malignant tissue visible and to re-treat any that does exist, without changing out any equipment.

10. The system of claim 1, wherein the system has the ability to tailor the shape, size and orientation of the eradicated tissue volume to whatever the shape, size and orientation of the mapped tumor(s). This can include multifocal tumors.

11. The ability to introduce and use optical energy from a transurethral probe creating a high optical contrast condition within the prostate tissue if tumors are present. Optical detectors mounted in the transrectal probe detect those contrast conditions. Acquired data is used for optical analysis to determine the presence of small multifocal tumors.

12. The system of claim 1, wherein the disclosed laser ablation process is not limited to prostate cancer but is applicable (with modified delivery) to other cancer sites, for example, liver cancer.
